SEQUENCE LISTING (12 sequences)

SEQ ID NO: 1 - protein, 37 aa, Artificial Sequence
  synthetic human cathelicidin protein LL-37
LLGDFFRKSKEKIGKEFKRIVQRIKDFLRNLVPRTES

SEQ ID NO: 2 - protein, 16 aa, Artificial Sequence
  synthetic human cathelicidin protein LL-37 active fragment
FKRIVQRIKDFLRNLV

SEQ ID NO: 3 - protein, 25 aa, Artificial Sequence
  synthetic human cathelicidin protein LL-37 active fragment
IGKEFKRIVQRIKDFLRNLVPRTES

SEQ ID NO: 4 - DNA, 20 bp, Artificial Sequence
  synthetic PCR sense primer BMPRIA
atgcgtgaggttgtgtgtgt

SEQ ID NO: 5 - DNA, 20 bp, Artificial Sequence
  synthetic PCR antisense primer BMPRIA
acccagagcttgactggaga

SEQ ID NO: 6 - DNA, 20 bp, Artificial Sequence
  synthetic PCR sense primer BMPRIB
aggtcgctatggggaagttt

SEQ ID NO: 7 - DNA, 20 bp, Artificial Sequence
  synthetic PCR antisense primer BMPRIB
tagcaacctcccaaaggatg

SEQ ID NO: 8 - DNA, 20 bp, Artificial Sequence
  synthetic PCR sense primer BMPRII
ccatgaggctgactggaaat

SEQ ID NO: 9 - DNA, 20 bp, Artificial Sequence
  synthetic PCR antisense primer BMPRII
aggaccaatttttggcacac

SEQ ID NO: 10 - DNA, 19 bp, Artificial Sequence
  synthetic PCR sense primer LL-37/hCAP18
gcttttgcatcaggctcag

SEQ ID NO: 11 - DNA, 20 bp, Artificial Sequence
  synthetic PCR antisense primer LL-37/hCAP18
gggtagggcacacactagga

SEQ ID NO: 12 - protein, 9 aa, Artificial Sequence
  synthetic nine-residue propeptide
LEISSTCDA

(19) United States
(12) Patent Application Publication
(10) Pub. No.: US 2010/0310514 A1
(43) Pub. Date: Dec. 9, 2010

(54) Anti-Inflammatory Bacteria

(76) Inventors: Chi Hin Cho, Hong Kong (CN); Yangchao Chen, Hong Kong (CN); Ka Kei Wu, Hong Kong (CN); Ching Man Wong, Hong Kong (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/793,445

(22) Filed: Jun. 3, 2010

Related U.S. Application Data
(60) Provisional application No. 61/232,106, filed on Aug. 7, 2009.
(60) Provisional application No. 61/184,226, filed on Jun. 4, 2009.

(51) Int. Cl. A61K 35/74; C12N 1/20; A61P 35/00; A61P 1/00; C07H 21/04

(52) U.S. Cl. 424/93.2; 435/252.3; 536/23.7

(57) ABSTRACT

Lactic acid bacteria expression cathelicidin, and methods of their use, are provided.

### CROSS-REFERENCE TO RELATED PATENT APPLICATIONS

The present patent application claims benefit of priority to U.S. Provisional Patent Application No. 61/184,226, filed Jun. 4, 2009, and U.S. Provisional Patent Application No. 61/232,106, filed Aug. 7, 2009, each of which are incorporated by reference for all purposes.

### BACKGROUND OF THE INVENTION

Cathelicidin antimicrobial peptides are a family of polypeptides found in lysosomes in polymorphonuclear leukocytes (PMNs). Members of the cathelicidin family of antimicrobial polypeptides are characterized by a highly conserved region (cathelin domain) and a highly variable cathelicidin peptide domain. Cathelicidin peptides have been isolated from many different species of mammals. Cathelicidins were originally found in neutrophils but have since been found in many other cells including epithelial cells and macrophages activated by bacteria, viruses, fungi, or the hormone 1,25-D.

LL-37 is a human cathelicidin protein. The gene product is synthesized as a propeptide (designated as “hCAP-18”). See, e.g., Agerberth et al., Proc Natl Acad Sci USA 92:195-199 (1995) This propeptide is cleaved extracellularly to produce LL-37 (Gudmundsson et al., Eur J Biochem 238:325-332 (1996)), which has broad anti-microbial activity. See, e.g., Chromek, et al., Nature Medicine 12(6):636-641 (2006).

Human cathelicidin (LL-37) encoded in plasmids can promote ulcer healing in rat stomachs. The peptide by itself can increase gastric epithelial cells proliferation through the transforming growth factor a/epidermal growth factor receptor pathway. See, Yang, Y. H., et al., J. Pharmacol. Exp. Therap. 318:547-554 (2006). The anti-inflammatory action of cathelicidin on ulcerative colitis in mice has been shown. See, Tai, E. K. K., et al., Exp. Biol. Med. 232, 799-808 (2007).

### BRIEF SUMMARY OF THE INVENTION

The present invention provides for lactic acid bacteria transformed to secrete biologically active cathelicidin. In some embodiments, the lactic acid bacteria is selected from the group consisting of Lactococcus sp., Lactobacillus sp., and Bifidobacterium sp. In some embodiments, the bacteria is Lactococcus.

In some embodiments, expression of the cathelicidin is under the control of an inducible promoter.

In some embodiments, the bacteria comprises a nucleic acid coding sequence for the cathelicidin and the coding sequence has been codon-improved for the species of the bacteria.

In some embodiments, the cathelicidin is substantially identical to (e.g., at least 80% identical to) SEQ ID NO:1 or SEQ ID NO:2.

The present invention also provides for methods of reducing inflammation in the gastrointestinal tract of a mammal by administering an amount of the lactic acid bacteria as described herein sufficient to reduce inflammation in the gastrointestinal tract of the mammal.

In some embodiments, 10⁸ to 10¹² CFU of the bacteria are administered to the mammal daily. In some embodiments, greater than 10¹⁰ CFU of the bacteria are administered to the mammal daily. In some embodiments, the bacteria is administered for at least three days. In some embodiments, the bacteria is administered daily for no more than seven, ten or 14 days.

In some embodiments, the bacteria secrete cathelicidin in an amount of at least 1 pg/day under defined in vitro culture conditions.

In some embodiments, the lactic acid bacteria is selected from the group consisting of Lactococcus sp., Lactobacillus sp., and Bifidobacterium sp. In some embodiments, the bacteria is Lactococcus.

In some embodiments, the mammal has colitis or bowel inflammatory disease. In some embodiments, the mammal has Crohns' disease. In some embodiments, the mammal has colorectal or gastric cancer. In some embodiments, the mammal has a condition selected from the group consisting of: gastritis, gastric ulcers or gastroesophageal reflux disease (GERD).

In some embodiments, expression of the cathelicidin is under the control of an inducible promoter and the method further comprises administering a sufficient amount of an inducer to induce expression of the promoter in the gut of the animal.

In some embodiments, the bacteria is administered orally.

In some embodiments, the bacteria is co-administered with a cathelicidin peptide.

In some embodiments, the cathelicidin is substantially identical (e.g., at least 80% identical to) SEQ ID NO:1 or SEQ ID NO:2.

In some embodiments, the mammal is a human.

The present invention also provides for a food product comprising an amount of the lactic acid bacteria as described herein sufficient to reduce inflammation in the gastrointestinal tract of a mammal.

In some embodiments, the food product is a beverage or a semi-solid product. In some embodiments, the semi-solid product is yogurt.

In some embodiments, expression of the cathelicidin is under the control of an inducible promoter and the food product further comprises a sufficient amount of an inducer to induce expression of the promoter.

The present invention also provides for an isolated nucleic acid comprising a nucleic acid coding sequence for a biologically active cathelicidin, wherein the coding sequence has been codon-improved for expression in a lactic acid bacteria. In some embodiments, the nucleic acid further comprises a promoter operably linked to the coding sequence. In some embodiments, the promoter is an inducible promoter. In some embodiments, the coding sequence comprises at least one codon improved for expression in Lactococcus sp., Lactobacillus sp., or Bifidobacterium sp.

### DEFINITIONS

The term “isolated,” when applied to a nucleic acid or protein, denotes that the nucleic acid or protein is essentially free of other cellular components with which it is associated in the natural state. It is optionally in a homogeneous state and can be in, e.g., a dry or aqueous solution. Purity and homogeneity are typically determined using analytical chemistry techniques such as polyacrylamide gel electrophoresis or high performance liquid chromatography.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N.Y. (1984)).

It is further recognized that non-naturally-occurring amino acids can be used to replace or more amino acids in a biologically active protein. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “identical” or percent “identity,” in the context of describing two or more polynucleotide or amino acid sequences, refer to two or more sequences or subsequences that are the same or have a specified percentage of amino acid residues or nucleotides that are the same (i.e., a sequence has 80% identity, preferably 85%, 90%, 91%, 92%, 93, 94%, 95%, 96%, 97%, 98%, 99%, or 100% identity, to a reference sequence, e.g., SEQ ID NO:1 or SEQ ID NO:2), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An algorithm suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

A “promoter” is an array of nucleic acid control sequences that direct transcription of a nucleic acid.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

“Cathelicidin” as used herein, refers to a member of the cathelicidin family of antimicrobial polypeptides, whose pro-proteins, when naturally occurring, are characterized by a highly conserved region (cathelin domain) and a highly variable cathelicidin peptide domain. This latter domain is biologically active, i.e., has antiLmicrobial activity. A variety of cathelicidin polypeptides have been described including a human sequence (CAP 18, with “LL-37” referring to the active, cleaved portion of the protein), a mouse protein (CRAMP), a sheep protein (SC5), a bovine protein (Bac5), a pig protein (PR-39) and a family of fish proteins (see, e.g., U.S. Pat. No. 7,351,693). In additional to the C-terminal peptides generated endogenously, smaller fragments of the C-terminal region of cathelicidin polypeptides have been described to have activity. See, e.g., US Patent Publication No. 2009/0088382.

Cathelicidins have the ability to bind to polyclonal antibodies generated against the prototype proteins SEQ ID NOs: 1 or 2. Under designated immunoassay conditions, the specified antibodies bind to cathelicidin by at least two times the background and do not substantially bind in a significant amount to other proteins present in the sample. For example, polyclonal antibodies raised to a protein consisting of SEQ ID NO:1 or 2, splice variants, or portions thereof, can be selected to obtain only those polyclonal antibodies that are specifically immunoreactive with SEQ ID NO:1 or 2 and not with other proteins, except for polymorphic variants and alleles of SEQ ID NO:1 or 2. This selection may be achieved by subtracting out antibodies that cross-react with molecules such as non-human cathelicidin orthologs. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

“Biologically-active” cathelicidin polypeptides as described herein have antimicrobial activity. Exemplary biologically active LL-37 and mouse CRAMP polypeptides have a minimal inhibitory concentration of 8 μM or lower (lower values represent higher activity), as measured in the assay set forth in Chromek, et al., Nature Medicine 12(6):636-641 (2006).

“Lactic acid bacteria” refer to a Glade of Gram-positive, low-GC, acid-tolerant, generally non-sporulating, non-respiring rod or cocci that are associated by their common metabolic and physiological characteristics. These bacteria, usually found in decomposing plants and lactic products, produce lactic acid as a major metabolic end-product of carbohydrate fermentation. Exemplary lactic acid bacteria genera include: Lactobacillus, Leuconostoc, Pediococcus, Lactococcus, Streptococcus, and Bifidobacteria.

### BRIEF DESCRIPTION OF THE DRAWINGS

FIG. 1. In vitro effects of LL-37 on gastric cancer cell proliferation. A, Incubation with LL-37 for 24 h inhibited the DNA synthesis of AGS and TMK1 cells. B, RT-PCR showed the mRNA expression of LL-37/hCAP18 in AGS and TMK1 cells. C, Knockdown or induction of LL-37/hCAP18 expression increased or suppressed TMK1 cell proliferation. 2×10⁴ TMK1 cells were transfected with 20 pmol/L LL-37/hCAP-18-siRNA or control siRNA or treated with or without 1 μmol/L 1a,25-dihydroxyvitamine D₃ (VD₃) for 24 h. Cell proliferation and LL-37/hCAP18 mRNA expression were determined 48 h post-transfection or post-treatment. D, LL-37 treatment induced accumulation of G₀/G₁-phase cells in TMK1. TMK1 cells were treated without or with 10 μg /ml or 20 μg /ml LL-37 for 24 h, and their DNA contents were determined by flow cytometry analysis. The results are the representative of 3 independent experiments. *, P<0.05; **, P<0.01, significantly different from respective control group.

FIG. 2. In vivo effects of LL-37 on the growth of gastric cancer xenograft. A, 4 consecutive injections of 40 μg LL-37 on alternate days significantly reduced the volume of TMK1 cancer xenograft transplanted in nude mice by 49%. B, at the end of the experiment, the tumors were excised and weighted. The tumor mass was significantly reduced by 40% in the LL-37-injected group.

FIG. 3. The effects of LL-37 on Smad signaling. A, LL-37 time- and concentration-dependently increased Smad1/5 but suppressed Smad2/3 phosphorylation. The expression of total Smad1 and Smad2/3 were unaffected by LL-37 treatment. The results are the representative of 3 independent experiments. B, real-time PCR results show that LL-37 (2 μg /ml) time-dependently increased the mRNA expression of Smad6 and Smad7 in TMK1 cells. *, P<0.05; **, P<0.01, significantly different from respective control group.

FIG. 4. The up-regulation and functional involvement of p21^{Waf1/Cip1} in the inhibitory effect of LL-37 on TMK1 cell proliferation. A, treating the cells with LL-37 (20 μg/ml) for 18 h specifically induced the mRNA expression of p21^{Waf1/Cip1} but not p15^{Ink4a} or p27^{Kip1} B, 24-hour treatment of LL-37 markedly increased p21^{Waf1/Cip1} protein expression. C, p21^{Cip1/Waf1}-siRNA down-regulated p21^{Waf1/Cip1} mRNA expression induced by LL-37 (20 μg/ml). Cells were transfected for 30 h followed by 24-hour treatment with LL-37 (20 μg/ml). Messenger RNA was extracted 30 h post-transfection. D, p21^{Cip1/Waf1}-siRNA abrogated the anti-mitogenic action of LL-37 (20 μg/ml). **, P<0.01, significantly different from respective control group; ^{††, P<}0.01, significantly different from control siRNA-transfected group treated with LL-37.

FIG. 5. The involvement of BMP receptor in the anti-mitogenic effect of LL-37 on TMK1 cells. A, original gel picture shows the mRNA expression of BMP receptor type IA (BMPRIA), BMPRIB, and BMPRII as determined by RT-PCR. B, Transfection efficiency was determined by transfecting TMK1 cells with fluorescent-labeled RNA duplex. C, BMPRII-siRNA significantly reduced the mRNA expression of BMPRII. D, siRNA-mediated knockdown of BMPRII abolished the increase in Smad1/5 phosphorylation and p21^{Waf1/Cip1} expression. 1×10⁵ cells were transfected with 100 pmol BMPRII-siRNA for 24 h followed by 24-hour treatment with LL-37 (20 μg/ml) before protein extraction. The results are the representative of 3 independent experiments. E, knockdown of BMPRII partially abolished the anti-mitogenic effect of LL-37 (20 μg/ml) on TMK1 cells. F, Real-time PCR revealed that treating TMK1 cells with LL-37 (20 μg/ml) for 8 h significantly increased the expression of BMP4 and BMP7. **, P<0.01, significantly different from respective control group; ^{††}, P<0.01, significantly different from control siRNA-transfected group treated with LL-37.

FIG. 6. The effects of LL-37 on the activity of 20S proteasome. A, The trypsin-like, chymotrypsin-like and caspase-like activities of proteasome in TMK1 cells treated with or without 20 μg /ml LL-37 for 8 h were assayed using a chemiluminescence-based method. MG-132 (1 μmol/L) was used as a positive control. B, 4-h treatment with MG -132 (1 μmol/L) significantly up-regulated the mRNA expression of BMP4. C, 4-h treatment with MG -132 (1 μmol/L) significantly increased Smad1/5 phosphorylation.

FIG. 7. Expression of LL-37/hCAP-18 and p21^{Waf1/Cip1} mRNA in human gastric cancer tissues. A, LL-37/hCAP-18 and p21^{Waf1/Cip1} mRNA expression in each cancer tissue sample were compared with the corresponding surrounding non-malignant tissue as determined by real-time PCR. Results of normal-cancer tissue pairs from 10 patients show significant down-regulation of LL-37/hCAP-18 and p21^{Waf1/Cip1} in gastric cancer tissues. B, a statistically significant positive correlation between the expression of LL-37/hCAP-18 and p21^{Waf1/Cip1} was found in the tissue pairs.

FIG. 8 Experimental protocols of this study. Wild-type and Cnlp−/− mice were fed with 3% DSS for 5 days to induce acute colitis. The protective effects of mCRAMP were determined by supplement with (A) daily administration of synthetic peptide or (B) a single administration of mCRAMP-expressing plasmid.

FIG. 9 Colonic mCRAMP expression induced by administration of mCRAMP-expressing plasmid. mCRAMP-expressing plasmid pcDNA3.1/mCRAMP (200 μg per mouse) was administered intra-rectally to the wild-type mice. Colonic tissues were collected after 2 days. Western blot was performed on the colonic protein extracts to detect the expression of mCRAMP.

FIG. 10 Representative histologic findings in wild-type and Cnlp−/− mice. Colonic tissues from mice with different DSS and mCRAMP treatment were fixed in 10% formalin. Sections (5 μm) were prepared and stained with hematoxylin-eosin (original magnification 100×). 5-day DSS treatment entirely destroyed mucosal structure and reduced crypt formation in Cnlp−/− mice. Peptide or plasmid treatment preserved the mucosal crypt structure.

FIG. 11 Effects of mCRAMP supplement on inflammatory markers in normal and colitis mice. (A) Colon length in normal and colitis mice was measured from the colo-cecal junction to the anal verge on Day 5. (B) MPO activity was calculated as enzyme units and was normalized by the amount of protein. The amounts of (C) IL-1β and (D) TNF-α in protein homogenates prepared from colonic tissue were measured by ELISA. The level of cytokines was expressed as pg of particular cytokine per mg of protein. Values are mean ±S.E. (n=8 per group). *P<0.05 and **P<0.01 when compared with the indicated groups.

FIG. 12 Effects of mCRAMP supplement on the activity of MMP-9 in colonic tissues of colitis mice. (A) Colonic tissue homogenates from wild-type and Cnlp−/− mice were analyzed by gelatin zymography. Each lane represents 15 μg protein from an individual mouse with or without mCRAMP supplement. Gelatinolytic bands of MMP-9 are indicated by an arrow. (B) Colonic MMP-9 activity was calculated semi-quantitatively in a multianalyzer. Higher activity was shown by increased intensity of colorless bands. **P<0.01 when compared with other groups.

FIG. 13 Effects of mCRAMP supplement on apoptosis in colonic tissues from normal and colitis mice. Results were expressed as the total number of apoptotic cells per field (original magnificent 200×). Values are mean ±S.E. (n=8 per group). *P<0.05 when compared with the indicated group.

FIG. 14 Effects of mCRMAP supplement on fecal microflora populations in normal and colitis mice. (A) Aerobic microflora. (B) Anaerobic microflora. The quantification was checked at the end of experiment (i.e. Day 5) in freshly passed stools. Amount of microflora was calculated as log10 of the total number of CFUs found per dry weight (gram) of feces. Values are mean ±S.E. (n=8 per group). *P<0.05 when compared with the indicated groups.

FIG. 15 Effects of mCRAMP supplement on the mucus-secreting layer and mucin expression in normal and colitis mice. (A) The length of the mucus-secreting layer and the total mucosal thickness were measured. (B) MUC1, (C) MUC2, (D) MUC3 and (E) MUC4 gene expression were determined by real-time PCR and standardized against the expression of β-actin. Values are mean ±S.E. (n=8 per group). *P<0.05 and **P<0.01 when compared with the indicated groups.

FIG. 16 illustrates MPO activity of mice treated as described in the Examples. a: p<0.05 compare with DSS Ctrl. b: p<0.01 compare with DSS Ctrl. c: p<0.01 compared with water Ctrl. d: p<0.01 compared with N4. Abbreviations as follows: water Ctrl: water control (n=8); DSS Ctrl: dextran sodium sulfate (n=8); N: DSS+Lactococcus lactis NZ3900 (n=7); N4: DSS+NZ3900 transformed with cathelicidin (n=7); N41: DSS+NZ3900 transformed with cathelicidin plus nisin (0.25 ng/mL) induction (n=8).

FIG. 17 illustrates MPO activity of mice treated as described in the Examples as follows: C: water only (n=8); N: water+Lactococcus lactis NZ3900 (n=4); N4: water+NZ3900 transformed with cathelicidin gene (n=4); and N41: water+NZ3900 transformed with cathelicidin gene plus nisin induction (0.25 ng/mL) (n=4).

FIG. 18 shows the effect of LL-37 and fragments thereof, on TMK1 cell proliferation at the concentrations indicated.

FIG. 19 shows crypt loss in mice following various treatments as set forth for FIG. 16. *p<0.05 compared with DSS ctrl.

FIG. 20. Disease activity index of mice (based on weight loss, stool consistency and bleeding) after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1 x 10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. *p<0.05, ***p<0.001 compared with DSS; ̂p<0.01 compared with DSS+N0I; +++p<0.001 compared with DSS+CMC-Na. (N=9-10 mice).

FIG. 21. Ratio of mucus secreting layer to mucosal thickness after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. *p<0.05, **p<0.01, ***p<0.001 compared with DSS; @p<0.05 compared with DSS+NO; ̂p<0.001 compared with DSS+N0I. (N=8-10 mice).

FIG. 22. Crypt score (based on mucosal crypt damage) after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. *p<0.05, ***p<0.001 compared with DSS; @p<0.001 compared with DSS+N0I; +p<0.05 compared with DSS+CMC-Na. (N=8-9 mice).

FIG. 23. Number of apoptotic cell per field after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) for or 600 mg/kg sulfasalazine (SASP) suspended in sodium carboxymethyl cellulose (CMC-Na) (given orally once daily) for 7 days. ***p<0.001 compared with DSS; @p<0.001 compared with DSS+N0I; ̂p<0.001 compared with DSS+8 log cfu N4I. (N=8-12 mice).

FIG. 24. Basal myeloperoxidase (MPO) activity in the colonic mucosa after treatment (given orally once daily) with 1×10¹⁰ cfu Lactococcus lactis NZ3900 without (N0) or with addition of nisin (0.25 ng/mL) (N0I) or transformed with mCRAMP without (N4) or with nisin (0.25 ng/mL) induction (N4I) for 7 days. (N=4-5 mice).

FIG. 25. Myeloperoxidase (MPO) activity after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (NOI) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. ***p<0.001 compared with DSS, ̂p<0.01 compared with DSS+N0I. (N=8-12 mice).

FIG. 26. Basal malondialdehyde (MDA) level after treatment (give orally once daily) with 1×10¹⁰ cfu Lactococcus lactis

NZ3900 without (N0) or with addition of nisin (0.25 ng/mL) (N0I) or transformed with mCRAMP without (N4) or with nisin (0.25 ng/mL) induction (N4I) for 7 days. (N=4-5 mice).

FIG. 27. Malondialdehyde (MDA) level after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (NOT) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. **p<0.01 compared with DSS; ++p<0.01 compared with DSS+CMC-Na. (N=9-13 mice).

FIG. 28. Basal number of aerobes per dry weight of feces after treatment (give orally once daily) with 1×10¹⁰ cfu Lactococcus lactis NZ3900 without (N0) or with addition of nisin(0.25 ng/mL) (N0I) or transformed with mCRAMP without (N4) or with nisin (0.25 ng/mL) induction (N4I) for 7 days. (N=4-5 mice).

FIG. 29. Number of aerobes per dry weight of feces after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. *p<0.05, **p<0.01 compared with DSS. (N=9-12 mice).

FIG. 30. Basal number of anaerobes per dry weight of feces after treatment (given orally once daily) with 1×10¹⁰ cfu Lactococcus lactis NZ3900 without (N0) or with addition of nisin (0.25 ng/mL) (N0I) or transformed with mCRAMP without (N4) or with nisin (0.25 ng/mL) induction (N4I) for 7 days. (N=4-5 mice).

FIG. 31. Number of anaerobes per dry weight of feces after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) (given orally once daily) for 7 days. *p<0.05, **p<0.01 compare with DSS. (N=9-12 mice).

FIG. 32. Basal colon length to body weight ratio after treatment (given orally once daily) with 1×10¹⁰ cfu Lactococcus lactis NZ3900 without (N0) or with addition of nisin (0.25 ng/mL) (N0I) or transformed with mCRAMP without (N4) or with nisin (0.25 ng/mL) induction (N4I) for 7 days. (N=4-5 mice).

FIG. 33. Colon length to body weight ratio after dextran sulfate sodium (DSS) administration (3%, w/v in drinking water) and treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) or 600 mg/kg sulfasalazine (SASP) suspended in sodium carboxymethyl cellulose (CMC-Na) (given orally once daily) for 7 days. ***p<0.001 compared with DSS; ̂p<0.01 compared with DSS+N4; +++p<0.001 compared with DSS+CMC-Na. (n=9-12 mice).

FIG. 34. Colon length to body weight ratio after dextran sulfate sodium (DSS) administration (3% w/v in drinking water) for 7 days followed by treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) for 4 days. *p<0.05, ***p<0.001 compared with DSS. (N=9 mice).

FIG. 35. Myeloperoxidase (MPO) activity after dextran sulfate sodium (DSS) administration (3% w/v in drinking water) for 7 days followed by treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) for 4 days. ***p<0.001 compared with DSS. (N=9-11 mice).

FIG. 36. Malondialdehyde (MDA) level after dextran sulfate sodium (DSS) administration (3% w/v in drinking water) for 7 days followed by treatment (given orally once daily) with 1×10¹⁰ cfu Lactococcus lactis NZ3900 without (N0) or with addition of nisin (0.25 ng/mL) (N0I) or 1×10⁸ cfu or 1×10¹⁰ cfu transformed with mCRAMP without (N4) or with nisin (0.25 ng/mL) induction (N4I) for 4 days. (N=8-9 mice).

FIG. 37. Study of the anticancer activity of Lactococcus lactis encoded with LL-37 using orthotopic gastric cancer model in nude mice. Oral administration of LL-37-encoding L. lactis (1×10¹⁰ CFU/mL) every other day for two weeks significantly reduced the tumor weight by 70% (P=0.0176 versus control group) and tumor weight/body weight by 71% (P=0.0092 versus control group) respectively. Animals in the control group received distilled water. The weights of tumors and body were measured on the day of sacrifice. Data are presented as mean ±SEM (n=4-5 mice).

### DETAILED DESCRIPTION

### I. Introduction

The present invention is based, in part, on the discovery that administration of probiotic lactic acid bacteria expressing cathelicidin is effective in ameliorating an induced colitis in a mouse disease model. The inventors have surprisingly found that probiotic bacteria can effectively deliver cathelicidin, which has anti-bacterial activity, in sufficient amount to ameliorate colitis. Cathelicidin also reduces gastric tumor growth. It is expected that the use of probiotic bacteria expressing cathelicidin will be effective in other gastrointestinal ailments, including those specifically described herein.

### II. Bacteria Expressing Cathelicidin

The present invention provides for lactic acid or other pro-biotic bacteria capable of colonizing the gut of a mammal recombinantly modified to express a biologically active cathelicidin polypeptide. Any number of lactic acid bacteria are known in the art and can be used in this invention. Exemplary lactic acid bacteria include, but are not limited to, Lactococcus sp. (including but not limited to, L. lactis, L. garivae, L. raffinolactis, L. plantarum), Lactobacillus sp. (including but not limited to, L. casei, L. palntarum, L. rhamnosus, L. acidophillus), or Bifidobacterium sp. (including but not limited to, B. longum, B. subtitle, B. bifidum, B. lactis). In some embodiments, the bacteria used is an accepted food or probiotic additive.

The bacteria of the invention will include an expression cassette encoding a biologically active cathelicidin polypeptide (as described herein). Expression of the cathelicidin polypeptide can be controlled by a promoter operably linked to the cathelicidin polypeptide coding sequence. Accordingly, expression cassettes of the invention can include a variety of components to regulate expression and localization of the polypeptides of the invention. For example, expression cassettes can include promoter elements, sequences encoding signal sequences, and a coding sequence for the polypeptide of interest.

A variety of promoters can be used to control cathelicidin expression, including constitutive and inducible promoters. Exemplary constitutive promoters include, but are not limited to, P59 (Van der Vossen et al., Appl. Environ. Microbiol. 58:3142-3149 (1992)), P23 (Elliot et al., Cell 36:211-219 (1984)), P32 or P44 (Drouault et al., Appl. Environ. Microbiol. 66 (2):588-598 (2000)) promoters).

Alternatively, expression can be under the control of an inducible promoter. Inducible promoters have the advantage of control over expression until a desired point and time. This is of particular value for expression of cathelicidin because of the antimicrobial activity of the polypeptide. In the case of the present invention, it can be desirable to have the cathelicidin expression under the control of an inducible promoter that is activated upon contact with a soluble inducer. Ideally, the inducer is at least relatively non-toxic and sufficiently stable for delivery to the gastrointestinal tract of a mammal. This allows for activation of cathelicidin expression only upon delivery of the inducer. Delivery of the inducer can occur simultaneously with delivery of bacteria, or can occur after, or in some embodiments before, delivery of the bacteria.

Exemplary inducible promoters include, but are not limited to, the Bacillus amylase (Weickert et al., J. Bacteriol. 171:3656-3666 (1989)) or xylose (Kim et al. Gene 181:71-76 (1996)) promoters as well as the Lactococcus nisin promoter (Eichenbaum et al, Appl. Environ. Microbiol. 64:2763-2769 (1998)). In the case of the nisin promoter, the inducer is nisin, a polycyclic peptide antibacterial with 34 amino acid residues commonly used as a food preservative. It contains the uncommon amino acids lanthionine (Lan), methyllanthionine (MeLan), didehydroalanine (Dha) and didehydroaminobutyric acid (Dhb). Nisin synthesis is described in, e.g., K. Fukase et al., Tetrahedron Lett. 29(7):795 (1988) and G. W. Buchman et al., J. Biol. Chem. 263(31):16260 (1988).

Another inducible promoter is the P170 promoter, which is a promoter from L. lactis. It is induced by pH decrease (pH<6) during transition from postexponential to stationary phases of glucose-grown cultures. It is self-inducible via lactic acid accumulation in the medium during growth (E Morello et al., J Mol Microbiol Biotechnol. 14(1-3):48-58 (2008)).

In addition, acid-induced promoters can be used. For example, promoters that are active under the relatively acidic conditions of the gut can be used. An exemplary acid-inducible promoter is the acid-induced RcfB promoter (Madson et al., Mol Microbiol 56(3) 735-746 (2005).

A variety of signal sequences are known to direct secretion of the cathelicidin polypeptides. Exemplary signal sequences include, e.g., usp45 (Van Asseldonk, Mol. Gen Genet. 240(3):428-434 (1993)), SP310 (Ravn et al., Microbiology 149: 2193-2201 (2003)) and Exp4 (Morello et al., J. Mol. Microbiol. Biotechhnol. 14 (1-3): 48-58 (2008). Signal sequences are typically located at the amino-terminus of a polypeptide.

In some embodiments, in addition to the biologically active amino acid sequence of the cathelicidin polypeptide, additional “pro-protein” amino acids are included. Generally, such sequences are elected such that the sequences are cleaved to generate an active protein at the desired site of delivery. For example, as discussed below, native cathelicidin is initially produced as a proprotein and subsequently cleaved into an active protein. Thus, in some embodiments, the hCAP-18 pro-protein is encoded by the expression cassette and LL-37 or another active fragment of hCAP-18 is generated by cleavage of the hCAP-18.

When designing a gene for improved expression in a host cell, it is desirable to design the gene such that its frequency of codon usage approaches the frequency of preferred codon usage of the host cell. The percent deviation of the frequency of preferred codon usage for a synthetic gene from that employed by a host cell is calculated first by determining the percent deviation of the frequency of usage of a single codon from that of the host cell followed by obtaining the average deviation over all codons.

The polynucleotide sequence encoding a particular polypeptide can be altered to coincide with the codon usage of a particular host. A “codon improved” coding sequence will comprise at least one codon that has been conformed to the host cell codon frequency compared to the native codon, i.e., the codon used in the genomic DNA of the species from which the coding sequence was derived. Ideally, a number of the codons are altered to the host cell preferred codon so as to improve expression in the host cell. For example, the codon usage of Lactobacillus can be used to derive a polynucleotide that encodes a polypeptide of the invention and comprises preferred Lactobacillus codons. The frequency of preferred codon usage exhibited by a host cell can be calculated by averaging the frequency of preferred codon usage in a large number of genes expressed by the host cell. In some cases, this analysis is limited to genes that are highly expressed by the host cell. Pouwels et al. (Nucleic Acids Res. 22:929-936 (1994)), for example, provides the frequency of codon usage by highly expressed genes exhibited by various Lactobacillus species. Lactococcus codon usage is described in, e.g., Gupta, et al., J. Biomolecular Structure and Dynamics 21(4): 1-9 (2004). Codon-usage tables are also available via the internet.

Any expression vector capable of controlling expression can be used, as desired.

Exemplary expression vectors include those described in, e.g., van de Guchte, et al., Appl Environ Microbiol. 55(1): 224-228 (1989); Jeong et al., Food Microbiology 23(5): 468-475 (2006); Sorving, et al., FEMS Microbiology Letters, 229(1): 119-126 (2006); U.S. Pat. No. 5,529,908; and US Patent Publication No. 2008/0286833. Commercial vectors include, e.g., the NICE® Expression System (BOCA Scientific, Boca Raton, Fla.), which uses the nisin promoter.

Any bacterial transformation methods can be used as known in the art. In some embodiments, the bacterial host will be rendered competent for transformation using standard techniques, such as the rubidium chloride method or electroporation (see, e.g., Wei, et al., J. Microbiol. Meth. 21:97-109 (1995).

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 2002)).

### III. Cathelicidin

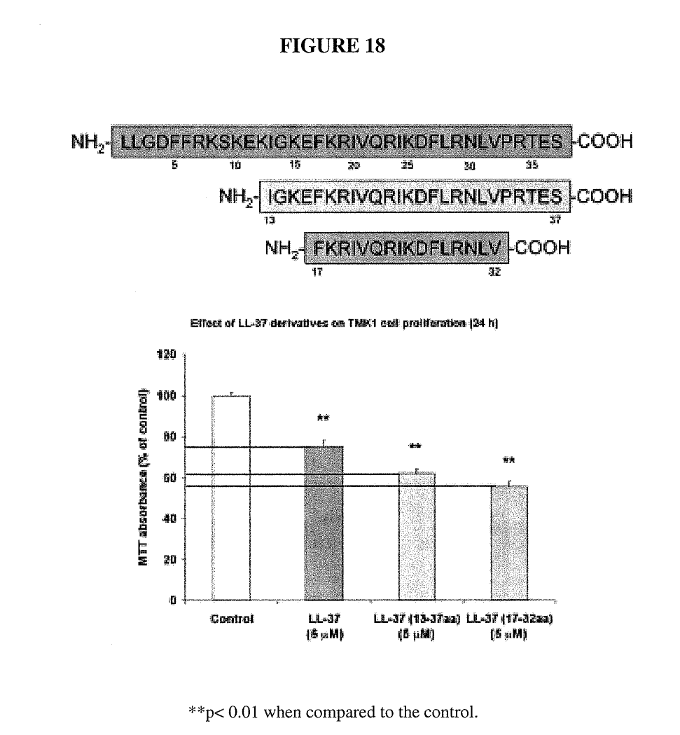

The present invention provides for expression of biologically active cathelicidin polypeptides in lactic acid bacteria or other probiotic bacteria as described herein. Biologically active cathelicidin polypeptides include biologically active LL-37 or active fragments thereof (see, e.g., US Patent Publication No 2009/0088382) or orthologs thereof from other species (e.g., m-CRAMP, etc.). LL-37 has the amino acid sequence: LLGDFFRKSKEKIGKEFKRIVQRIKDFLRNLVPRTES (SEQ ID NO:1). The present invention provides for active fragments of LL-37, e.g., fragments of at least 20, 25, 30, or 35 amino acids of LL-37, optionally further comprising one or more additional amino acid fused to the N- or C-terminus. For example, the inventors have found that peptides HN₂-IGKEFKRIVQRIKDFLRNLVPRTES—COOH and HN₂-FKRIVQRIKDFLRNLV—COOH have similar activity as LL-37. See, e.g., FIG. 18. Accordingly, the present invention provides for biologically active cathelicidin polypeptides comprising FKRIVQRIKDFLRNLV (SEQ ID NO:2). The present invention also provides for biologically active variants of SEQ ID NO:1 or SEQ ID NO:2, e.g., polypeptides comprising a SEQ ID NO:1 or SEQ ID NO:2 variant wherein 1, 2, 3, 4, 5, 6, 7, or more amino acids of SEQ ID NO:1 or SEQ ID NO:2 are replaced with a different amino acid, e.g., a conservative amino acid substitution.

### IV. Administration and Dosage

The bacteria of the present invention are useful for treating or ameliorating a number of disorders and diseases of the gastrointestinal tract. In some embodiments, the bacteria are administered to a mammal (e.g., a human) having, for example, colitis, inflammatory bowel disease or Crohns' disease, thereby ameliorating the disease or symptoms thereof. In some embodiments, the bacteria are administered to a mammal (e.g., a human) having, for example, colorectal or gastric cancer (including but not limited to gastric cancer related to Helicobacter infection), thereby ameliorating the cancer or symptoms thereof. In some embodiments, the bacteria are administered to a mammal (e.g., a human) having, for example, gastritis, gastric ulcers or gastroesophageal reflux disease (GERD), thereby ameliorating the disease or symptoms thereof. In any of these embodiments, the promoter controlling cathelicidin expression can be inducible, as described above. In these embodiments, the inducer (e.g., nisin in the case of a nisin-inducible promoter) can be administered simultaneously with the bacteria or separately. Optionally, the bacteria can be induced before administration. For example, the bacteria can be contacted with an inducer (e.g., nisin) prior to administration of the bacteria (using, e.g., about 10 ng/ml of nisin), incubated for a period of time to allow for induction or expression, and then administered. Optionally, excess inducer can be washed from the bacteria prior to administration of the bacteria.

The amount of the bacteria can be selected and determined depending on the purpose of use (e.g., disease to be treated or ameliorated). Exemplary dosages include, e.g., daily doses of about 10⁶-10¹⁴ CFU, e.g., 10⁸-10¹² CFU, 10¹⁰-10¹²CFU, 10⁸-10¹¹ CFU, etc. Such daily doses can be administered once daily or can be split into two or more doses within a one-day period. Optionally, smaller or larger doses can be used as desired (e.g., for general maintenance of health, etc.).

In some embodiments, dosage is determined by the amount of cathelicidin the bacteria can produce in defined in vitro culture conditions. In vitro cathelicidin production can be used to calculate the necessary amount of bacteria to administer to deliver an effective dosage. As explained in the Examples, the inventors have found that bacteria that express cathelicidin amounts as low as 1 pg/day in vitro under defined conditions are effective in vivo in mice for treatment of colitis. Such amounts are equivalent to at least 10 pg/day for human administration. Thus, in some embodiments, a sufficient amount of bacteria is administered to a human, wherein the amount of bacteria produce 1-5000 pg, e.g., 10-2000 pg of cathelicidin per day in vitro under defined conditions. The inventors have found that such levels are not sufficiently high to affect viability of the producing bacteria, thereby avoiding potential toxicity to the bacteria producing cathelicidin. “Defined in vitro culture conditions,” as used herein to describe assay conditions to determine the amount of cathelicidin polypeptide, refers to inoculation of an overnight culture of the bacteria in question at a 1/25 dilution into fresh media followed by a 30 minute incubation at 30° C. followed by induction of the promoter, if an inducible promoter is used, and further incubation with the inducer for about 3 hour until an OD₆₀₀ of ˜0.4 is reached. Cathelicidin amounts are then determined, e.g., by ELISA.

The dosages can be administered as long as necessary or desired. In some embodiments, the bacteria are administered daily for no more than 30, 20, or 10 days. In some embodiments, the dosage is administered for at least 4, 5, 6 or more days, e.g., 4-7, 4-10, 4-20, 4-30 days or longer, etc.

The bacteria of the present invention (encoding a cathelicidin protein) can be administered as desired or as known in the art. Administration of the bacteria of the invention is to the gastrointestinal tract. In some embodiments, the administration route is oral or rectal. In some embodiments, the bacteria of the invention are administered in a gel, suspension, aerosol spray, capsule, tablet, powder or semi-solid formulation (e.g., a suppository). Optionally, any food or beverage that can be consumed by a mammal (including but not limited to a human adult or a human infant or toddler) can be used to make formulations containing the bacteria of the present invention. Exemplary foods include those with a semi-liquid consistency to allow easy and uniform dispersal of the bacteria of the invention. However, other consistencies (e.g., powders, liquids, etc.) can also be used without limitation. Accordingly, such food items include, without limitation, dairy-based products such as cheese, cottage cheese, yogurt, and ice cream. Processed fruits and vegetables, including but not limited to those targeted for infants/toddlers, such as apple sauce or strained peas and carrots, are also suitable for use in combination with the bacteria of the present invention. In addition to foods targeted for human consumption, animal feeds can also be supplemented with the bacteria of the invention.

Alternatively, the bacteria of the invention may be used to supplement a beverage. Examples of such beverages include, without limitation, milk, fermented milk, fruit juice, fruit-based drinks, sports drinks, infant formula, follow-on formula, and toddler's beverage. Other beneficial formulations of the compositions of the present invention include the supplementation of animal milks, such as cow's milk.

Alternatively, the bacteria of the present invention can be formulated into pills or tablets or encapsulated in capsules, such as gelatin capsules. Tablet forms can optionally include, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge or candy forms can comprise the compositions in a flavor, e.g., sucrose, as well as pastilles comprising the compositions in an inert base, such as gelatin and glycerin or sucrose and acacia emulsions, gels, and the like containing, in addition to the active ingredient, carriers known in the art. The bacterial formulations may also contain conventional food supplement fillers and extenders such as, for example, rice flour.

In some embodiments, the bacteria of the invention are dried, for instance, freeze-dried, in the presence of a stabilizer to protect viability. Freeze dried preparations can be added to a food or a beverage by the consumer.

In some embodiments, the bacterial composition will further comprise a bovine (or other non-human) milk protein, a soy protein, a rice protein, betalactoglobulin, whey, soybean oil or starch.

### Examples

The following examples are offered to illustrate, but not to limit the claimed invention.

### Example 1

### Delivery of Cathelicidin Ameliorates Gastric Cancer

### Materials & Methods

Reagents and drugs—The synthetic LL-37 peptide was purchased from Invitrogen

(Carlsbad, Calif., USA). Antibodies for Smad1 and phospho-Smad1/5 were purchased from Cell Signaling Technology (Beverley, Mass., USA). Other primary antibodies were purchased from Santa Cruz Biotechnology (Santa Cruz, Calif., USA). 1α,25-dihydroxylvitamin D₃ was purchased from Wako Chemicals (Osaka, Japan). All other chemicals and reagents were purchased from Sigma (Sigma-Aldrich, St. Louis, Mo.) unless otherwise specified.

Clinical samples—Surgical specimens from 10 patients with gastric adenocarcinoma admitted to the Department of Surgery, Queen Mary Hospital, Hong Kong, for gastrectomy were obtained. Samples were collected before chemotherapy or any other form of treatment. There were 6 men and 4 women with a median age of 67. Five patients were diagnosed with intestinal-type adenocarcinoma, three with diffuse-type, and two with mixed-type. The study has been approved by the ethics committee, Queen Mary Hospital, Hong Kong.

Cell culture and viability assay—The human gastric adenocarcinoma cell line AGS was purchased from American Type Culture Collection (Manassas, Va., USA). The gastric adenocarcinoma cell TMK1 was obtained from Dr. Eiichi Tahara (University of Hiroshima, Hiroshima, Japan). AGS and TMK1 cells were maintained in RPMI 1640 (Invitrogen) containing 10% fetal bovine serum (Invitrogen), 100 U/mL penicillin G, 100 μg/mL streptomycin, and maintained at 37° C., 95% humidity, and 5% carbon dioxide. Cell viability was determined by lactate dehydrogenase release assay (Roche, Indianapolis, Ind., USA).

Cell proliferation assay and cell cycle analysis—Cell proliferation was measured as the amount of DNA synthesis using a modified [³H]-thymidine incorporation assay as previously described (Yang, Y. H., et al. (2006) J. Pharmacol. Exp. Ther, 318, 547-554). In brief, cells were treated with various doses of LL-37 for 24 h and then incubated with 0.5 μCi/mL [³H]-thymidine (Amersham Corporation, Arlington Heights, Ill. USA) for another 4 h. The final incorporation of [³H]thymidine into cells was measured with a liquid scintillation counter (LS-6500, Beckman Instruments, Inc., Pullerton, Calif.). For cell cycle analysis, cells were fixed with ice-cold 70% ethanol in phosphate buffered saline (PBS) followed by incubation with 50 μg/ml propidium iodide, 3.8 mmol/L sodium citrate, and 0.5 μg/ml RNase A at 4° C. for 3 h and analyzed by flow cytometry (Beckman Coulter, Fullerton, Calif., USA). The cell cycle phase distribution was calculated from the resultant DNA histogram using WinMDI 2.8 software.

Nude mice xenograft model—In order to evaluate the direct inhibitory action of LL-37 on cancer growth in vivo, a TMK1 gastric cancer xenograft model was adopted. In brief, TMK1 cells were trypsinized, collected and re-suspended in PBS (2×10⁷ cells/ml). Cell viability was confirmed to be above 95% based on trypan blue staining. Then 3×10⁶ TMK1 cells in 0.2 ml PBS were injected subcutaneously into the right flank or dorsal region of 4-to 6-week-old female BALB/c nu/nu mice. After inoculation, the mice were maintained under sterile condition and the size of tumor formed was measured using calipers every other days. Tumor volume (V) was estimated according to the following formula: V=L×W²/2, where L is the mid-axis length and W is the mid-axis width. After the tumors reached a mean size of 100-200 mm³ (on day 10), 40 μg of LL-37 dissolved in PBS was given to the nude mice every other day. Animals in the control group received equal volume of PBS. All treatments were administered by subcutaneous injections into 2 sites adjacent to the tumors in mice. At the end of the experiment, the mice were sacrificed and the tumors were excised and weighted.

Conventional and quantitative reverse transcription-polymerase chain reaction—The total RNA was isolated and cDNA was synthesized as previously described (Yang, Y. H., et al. (2006) J. Pharmacol. Exp. Ther, 318, 547-554). PCR was then performed using the following primer pairs: BMPRIA, sense primer 5′-ATGCGTGAGGTTGTGTGTGT-3′ and antisense primer 5′-ACCCAGAGCTTGACTGGAGA-3′ (product size 503 bp); BMPRIB, sense primer 5′-AGGTCGCTATGGGGAAGTTT-3′ and antisense primer 5′-TAGCAACCTCCCAAAGGATG-3′ (product size 599 bp); BMPRII, sense primer 5′-CCATGAGGCTGACTGGAAAT-3′ and antisense primer 5′-AGGACCAATTTTTGGCACAC-3′ (product size 563 bp); LL-37/hCAP18, sense primer 5′- GCTTTTGCATCAGGCTCAG-3′ and antisense primer 5′-GGGTAGGGCACACACTAGGA-3′ (product size 598 bp). Conditions for PCR were 94° C. for 5 min, 35 cycles of 94° C. for 30 sec, 55° C. for 30 sec and 72° C. for 1 min. The final extension step was at 72° C. for 7 min. The PCR products were then electrophoresed on a 1.0% agarose gel containing 0.5 μg/mL ethidium bromide. For semi-quantitation, real-time PCR was performed with specific pre-designed primer set purchased from Qiagen (Valencia, Calif., USA) or SuperArray Bioscience (Frederick, Md., USA) with b-actin as an internal control. Conditions for quantitative PCR were 94° C. for 5 min, 40 cycles of 94° C. for 30 sec, 55° C. for 30 sec and 72° C. for 30 sec. Quantitative PCR was carried out using iQ SYBR Green Supermix (Bio-Rad, Hercules, USA) and Multicolor Real-Time PCR Detection System (Bio-Rad). The results were analyzed using the comparative threshold cycle (C_{T}) method (Livak, K. J. and Schmittgen, T. D. (2001) Methods, 25, 402-408).

RNA interference—The expression of hCAP18/LL-37, BMPRII, and p21^{Waf1/Cip1} were lowered using validated target-specific siRNA molecules purchased from Qiagen. Two hundred picomoles of gene-specific or control siRNA was transfected into TMK1 cells at 40-60% confluence using Lipofectamine™ 2000 reagent (Invitrogen) according to the manufacturer's instructions. The transfection efficiency of siRNA was determined by transfection of fluorescent-labeled RNA duplex (Invitorgen) in TMK1 cells.

Immunoprecipitation and Western blot analysis—TMK1 cells were harvested in radioimmunoprecipitation buffer containing proteinase and phosphatase inhibitors as previously described (Yang, Y. H., et al. J. Pharmacol. Exp. Ther, 318, 547-554 (2006)). Equal amounts of protein (40 μg/lane) were resolved by SDS-PAGE, and transferred to Hybond C nitrocellulose membranes (Amersham Corporation). The membranes were probed with primary antibodies overnight at 4° C. and incubated for 1 h with secondary peroxidase-conjugated antibodies. They were developed with an enhanced chemiluminescence system (Amersham Corporation) and exposed to an X-ray film (FUJI Photo Film Co., Ltd., Tokyo, Japan).

Proteasome activity assay—Proteasome trypsin-like, chymotrypsin-like and caspase-like activities were determined by proteasome-Glo™ assay systems (Promega, Madison, Wis.). Briefly, cells were harvested in potassium phosphoate buffer (pH 7.4) containing 10 mM DTT, 1 mM PMSF and 0.1 mM EDTA. After sonication for 30 s on ice and centrifugation for 15 min at 12,000 g at 4° C., the supernatant was collected for proteasome activity assay according to the manufacturer's instructions. Chemiluminescent signals were detected by the ChemiDoc XRS documentation system (Bio-Rad).

Statistical analysis—Results were expressed as the mean ±SEM. Statistical analysis was performed with an analysis of variance (ANOVA) followed by the Turkey's t-test or Pearson correlation analysis. P values less than 0.05 were considered statistically significant.

### Results

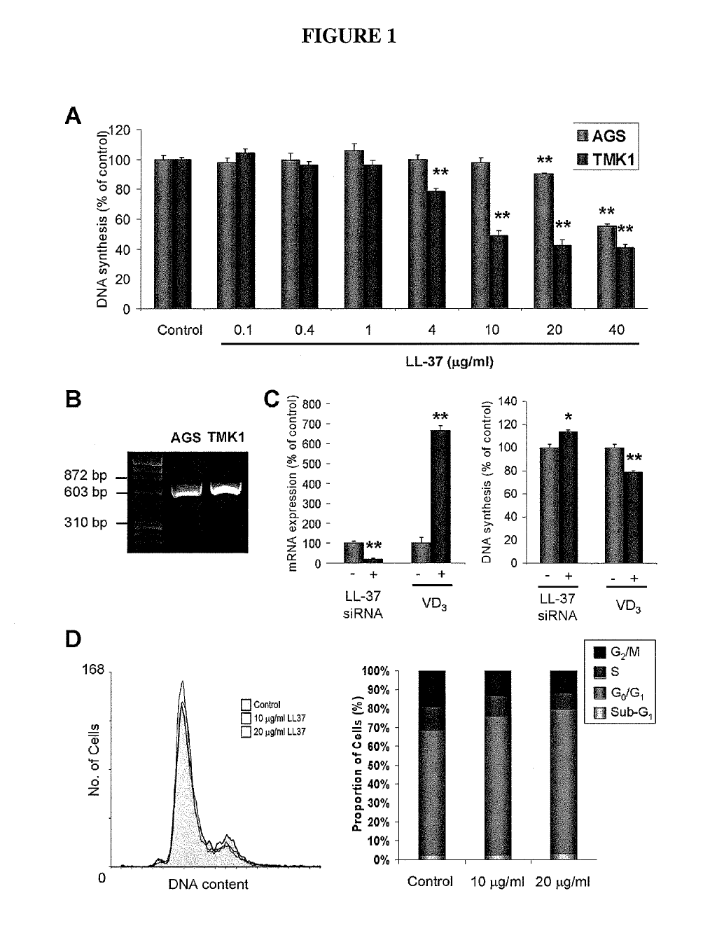

LL-37 inhibited gastric cancer cell proliferation and induced G₀/G₁-phase cell cycle arrest in vitro. To study the effect of LL-37 on proliferation of gastric cancer cells, we examined changes in [³H]thymidine incorporation in response to LL-37 treatment in cultured gastric cancer cell lines AGS and TMK1. As shown in FIG. 1A, 24 h-incubation with LL-37 significantly reduced [³H]thymidine incorporation in both cell lines in a concentration-dependent manner. At the dose of 20 μg/ml, LL-37 inhibited TMK1 cell proliferation by about 60%. AGS cells showed a similar response to LL-37 treatment. Nevertheless, AGS cells were less responsive than TMK1 cells, only showing 10% of inhibition at the dose of 20 μg/ml. The cell viability in all treatment groups was confirmed to be unaffected as determined by lactate dehydrogenase release assay. Results from RT-PCR also revealed that LL-37/hCAP18 mRNA was expressed in AGS and TMK1 (FIG. 1B). To further confirm the anti-mitogenic action of LL-37, the endogenous expression of LL-37 in TMK1 cells was suppressed by siRNA or induced by 1α,25-dihydroxylvitamin D₃, a known inducer of LL-37 (Liu, P. T., et al. (2006) Science, 311, 1770-1773). Results showed that knockdown or induction of endogenous LL-37 respectively increased or inhibited cell proliferation (FIG. 1C). These data supports that modulation of LL-37 expression at the physiological levels regulates cell proliferation. Further analysis by flow cytometry demonstrated that LL-37 induced an accumulation of TMK1 cells at the G₀/G₁ phase (66% in control group vs 77% in 20 μg /ml LL-37-treated group). A reciprocal reduction of proportion of cells in S and G₂/M phase was also observed in LL-37-treated cells (FIG. 1D). In particular, the total number of cells in S and G₂/M phases was reduced by 33% in the 20 μg/ml LL-37-treated group.

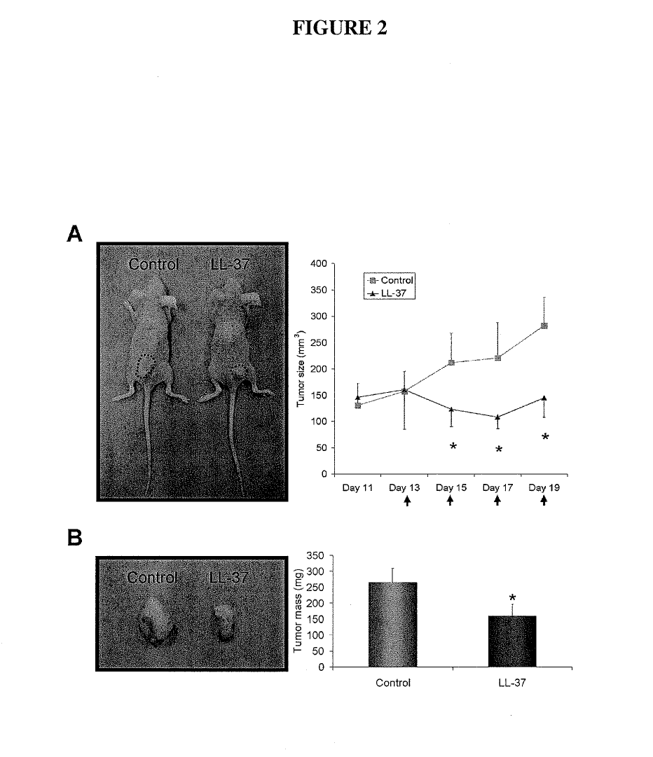

LL-37 inhibited the growth of gastric cancer xenograft in vivo. The direct anticancer activity of LL-37 in vivo was evaluated using a gastric cancer xenograft model in nude mice. After inoculation of TMK1 cells for 10 days, tumors reached a mean size of 150 mm³. Animals were thereafter randomized and treated as mentioned in Materials and Methods. Subcutaneous injection of LL-37 adjacent to the tumors on alternate days for a total of 4 injections significantly reduced the tumor volume by 49% when compared with control (FIG. 2A). At the time of excision, the tumor mass was reduced by 40% in the LL-37-injected group (FIG. 2B).

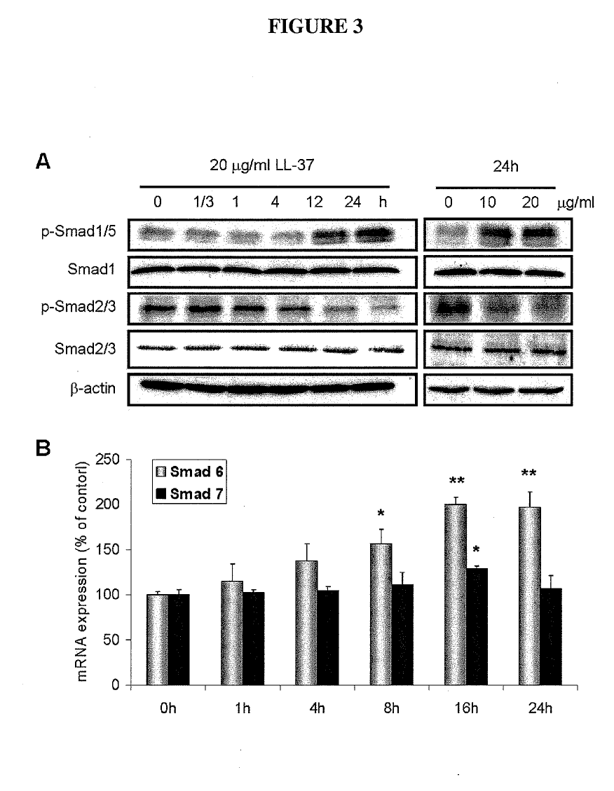

LL-37 increased Smad1/5 phosphorylation and Smad6/7 expression with concomitant suppression of Smad2/3 phosphorylation. TGF-β/BMP signaling has been shown to regulate gastric cancer cell proliferation (Wen, X. Z., et al. (2004) Biochem. Biophys. Res. Commun, 316, 100-106; Liu, P. T., et al. (2006) Science, 311, 1770-1773). To explore the relationship between LL-37 and TGF-β/BMP signaling, the phosphorylation levels of TGF-β-responsive Smad2/3 and BMP-responsive Smad1/5 were determined. Results showed that LL-37 significantly increased Smad1/5 phosphorylation but reduced Smad2/3 phosphorylation in a time- and dose-dependent manner. The protein expression levels of total Smad1 and Smad2/3 were not affected (FIG. 3A). The induction of Smad1/5 phosphorylation was observed as early as 12-h after treatment with LL-37. Moreover, the mRNA expression of Smad6 and Smad7, both of which are known to be inducible by BMP signaling (Nakao, A., et al. (1997) Nature, 389, 631-635; Wang, Q., et al. (2007) J. Biol. Chem, 282, 10742-10748), were significantly increased by LL-37 treatment (FIG. 3B).

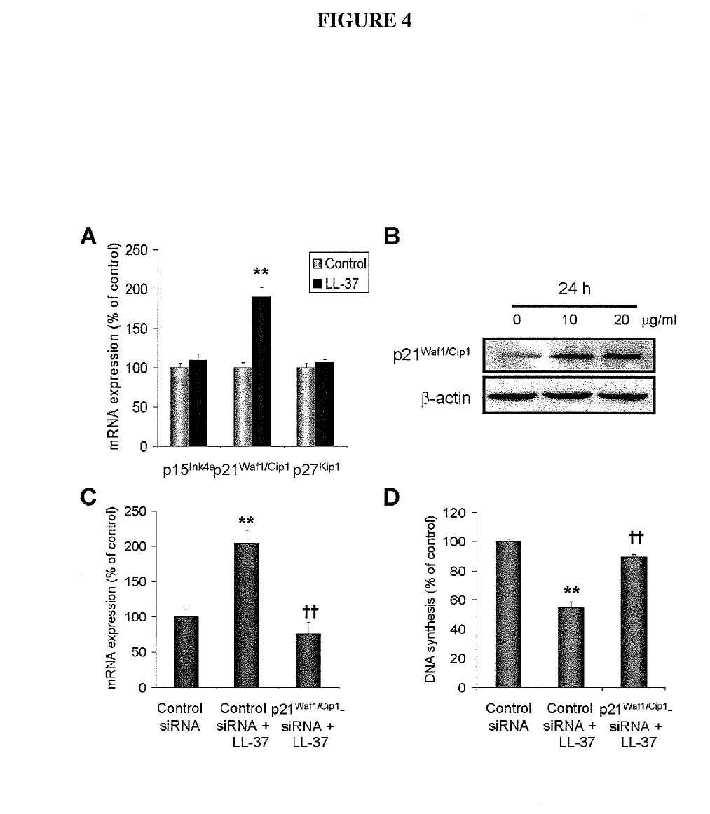

LL-37 induced cyclin-dependent kinase inhibitor p21^{Waf1/Cip1} but not p15^{Ink4a} or p27^{Kip1} expression. Activation of BMP signaling has been shown to inhibit cell cycle progression through the induction of specific CDK inhibitors such as p21^{Waf1/Cip1} and p27^{Kip1} (Wen, X. Z., et al. (2004) Biochem. Biophys. Res. Commun, 316, 100-106; Franzen, A. and Heldin, N. E. (2001) Biochem. Biophys. Res. Commun, 285, 773-781). In the present study, results showed that the activation of BMP signaling increased the mRNA (FIG. 4A) and protein (FIG. 4B) expression of p21^{Waf1/Cip1}. Nevertheless, the mRNA expression of p15^{Ink4a} or p27^{Kip1} was not significantly affected (FIG. 4A). To further substantiate the role of p21^{Waf1/Cip1} in the anti-mitogenic action of LL-37 in gastric cancer, the mRNA expression of p21^{Waf1/Cip1} induced by LL-37 was knocked down by siRNA. As shown in FIG. 4C, RNA interference reduced the p21^{Waf1/Cip1} mRNA expression in LL-37-treated cells almost back to the control level and partially cancelled the anti-mitogenic action of LL-37 (FIG. 4D).

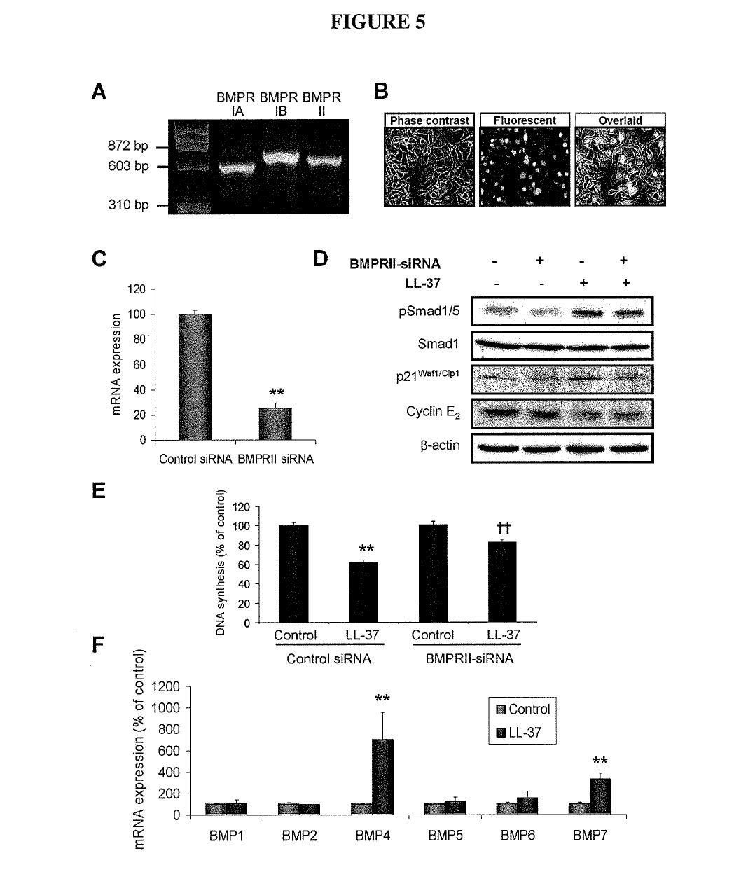

Knockdown of BMPRII attenuated the anti-mitogenic signaling induced by LL-37. Although the results presented so far indicated that LL-37 activated BMP signaling, whether this phenomenon is receptor-mediated had not yet been confirmed. We therefore determined the expression of BMPR, which normally exists as a heterodimer (Koenig, B. B., et al. (1994) Mol. Cell. Biol, 14, 5961-5974), in TMK1 cells. RT-PCR showed that TMK1 cells expressed BMPRIA, IB, and II (FIG. 5A). We therefore employed siRNA to knockdown BMPRII, which interacts with BMPRIA or IB to form a functional receptor. The siRNA transfection efficiency of TMK1 cells was more than 95% (FIG. 5B). BMPRII-siRNA significantly reduced the expression of BMPRII (FIG. 5C) and dampened the anti-mitogenic signals elicited by LL-37. In this respect, siRNA-mediated down-regulation of BMPRII abolished Smad1/5 phosphorylation and p21^{Waf1/Cip1} expression induced by LL-37 (FIG. 5D). The cyclin-dependent kinase (CDK) inhibitor p21^{Waf1/Cip1} is a known inhibitor of CDK₂/Cyclin E complex. Aside from induction of p21^{Waf1/Cip1} via activation of BMP signaling, our results indicate that LL-37 down-regulated cyclin E₂ via a BMPR-independent mechanism (FIG. 5D). To further confirm the involvement of BMP/p21^{Waf1/Cip1} pathway in the anti-mitogenic effect of LL-37, cell proliferation was determined in cells transfected with control or BMRRII siRNA in the absence or presence of LL-37. Results show that the inhibition of cell proliferation induced by LL-37 was partially reversed by the knockdown of BMPRII (FIG. 5E).

LL-37 induced BMP4 expression. The activity of BMP signaling relies on an intricate regulation of endogenous BMP ligand expression. In this respect, we demonstrated that TMK1 expressed all BMP ligands, from BMP1 to BMP7, except BMP3. Quantitative PCR further revealed that BMP4, and to a lesser extend BMP7, were significantly up-regulated after 8-h treatment with LL-37 (FIG. 5F). The expression of other BMPs, however, was not significantly altered.

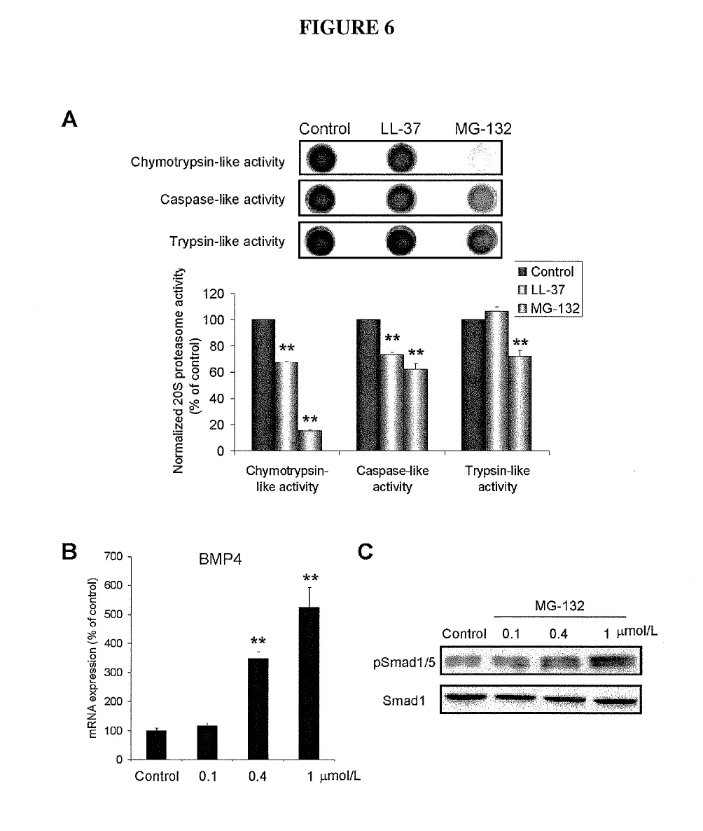

LL-37 inhibited chymotrypsin-like and caspase-like activity of proteasome. We previously demonstrated that the transcription of BMP4 and the activity of BMP signaling are under the regulation of the ubiquitin-proteasome pathway in gastric cancer cells (Wu, W. K., et al. (2008) Biochem. Biophys. Res. Commun, 371, 209-214). Here we demonstrated that treating TMK1 cells for 8 h significantly reduced the chymotrypsin-like and caspase-like activity but not the trypsin-like activity of 20S proteasome (FIG. 6A). Moreover, treating the cells with proteasome inhibitor MG-132 dose-dependently increased BMP4 mRNA expression (FIG. 6B) and Smad1/5 phosphorylation (FIG. 6C).

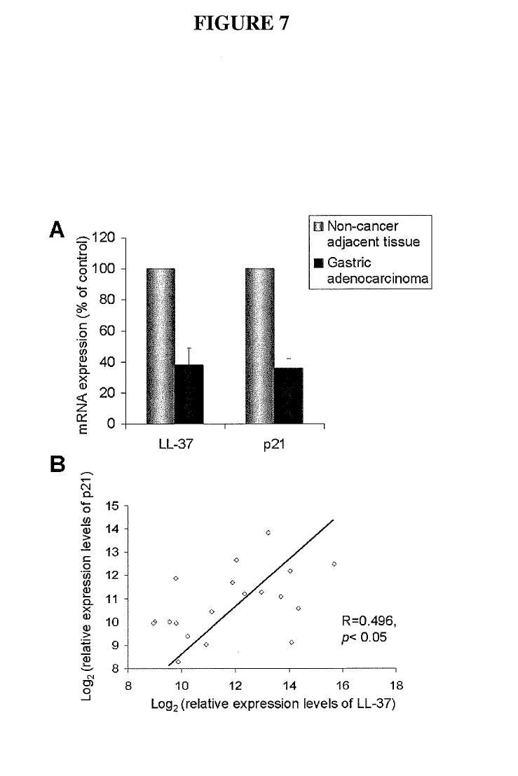

Both LL-37/hCAP18 and p21^{waf1/cip1} mRNA were down-regulated in human gastric cancer tissue. To determine whether the results derived from the cell line experiments we did so far are of clinical relevance, we compared LL-37/hCAP18 and p21^{Waf1/Cip1} mRNA expression in normal and cancerous gastric tissues obtained from human gastric cancer patients by quantitative RT-PCR. Results showed that both LL-37/hCAP18 and p21^{Waf1/Cip1} mRNA levels were significantly down-regulated in gastric cancer tissues, in which their expression levels were nearly down-regulated by 60% when compared with the surrounding non-malignant tissues (FIG. 7A). Moreover, there was a moderate but statistically significant correlation between the expression of LL-37/hCAP18 and p21^{Waf1/Cip1} (FIG. 7B), suggesting that the expression of p21^{Waf1/Cip1} might be partially governed by LL-37/hCAP18 in human gastric cancer.

### Discussion

In mammals, a plethora of host defense peptides, such as β-defensins and cathelicidins, serve important innate immune functions by providing first-line defense against infection. These peptides are expressed in circulating immune cells and on epithelial surfaces of the skin and gastrointestinal tract and are up-regulated on infection (Hase, K., et al. (2003) Gastroenterology, 125, 1613-1625). Later studies reveal that these peptides play other crucial roles such as modulation of inflammation and promotion of tissue repair (Mookherjee, N., et al. (2006) J. Immunol, 176, 2455-2464; Yang, Y. H., et al. (2006) J. Pharmacol. Exp. Ther, 318, 547-554; Tai, E. K., et al. (2007) Exp. Biol. Med, 232, 799-808). A recent report shows that LL-37, the only cathelicidin in human, seems to be down-regulated in gastric cancer tissue (Hase, K., et al. (2003) Gastroenterology, 125, 1613-1625).

In the present study, we demonstrate that the synthetic LL-37 peptide significantly lowers gastric cancer cell proliferation by delaying G₁-S transition during cell cycle progression in vitro and inhibits the growth of gastric cancer xenograft in vivo. Knockdown or induction of the endogenous LL-37 expression by siRNA or 1α,25-dihydroxyvitamin D₃, respectively, increases or suppresses gastric cancer cell proliferation. These findings indicate that LL-37 is a negative regulator of cell growth in gastric cancer. To date, there is only one report showing that LL-37 has anti-mitogenic action in cancer cells. In that study, LL-37 inhibited the growth of two human oral epidermoid carcinoma cell lines (Li, X., et al. (2006) J. Am. Chem. Soc, 128, 5776-5785). This finding is in line with our conclusion that LL-37 may function as a tumor suppressor in human cancers. Contrary evidence, nevertheless, also exists in the literature in which LL-37 promotes the proliferation of cultured breast cancer cells and is highly expressed in high-grade breast cancer tissues (Heilborn, J. D., et al. Int. J. Cancer, 114, 713-719). Without intending to limit the scope of the present invention, we speculate that the anti-mitogenic action of LL-37 in cancer may be highly tissue-specific.

Activation of BMP signaling has been shown to inhibit gastric cancer cell proliferation. For instances, BMP2 inhibits gastric cancer growth and induces G₀/G₁ cell cycle arrest through induction of the CDK inhibitor p21^{Waf1/Cip}. A later study further unraveled that BMP2 was down-regulated in gastric cancer via epigenetic silencing. Moreover, a recent study demonstrated that abolishment of BMP signaling by conditional inactivation of BMPRIA in mice promotes gastric cancer formation (Bleuming, S. A., et al. (2007) Cancer Res, 67, 8149-8155). All these findings implicated that BMP signaling may play a suppressive role in gastric carcinogenesis. In this respect, our results indicate that BMP signaling was activated by LL-37, in which a time- and dose-dependent increase in BMP-responsive Smad1/5 phosphorylation could be observed. Moreover, LL-37 induced the expression of BMP-responsive transcriptional targets. To this end, p21^{Waf1/Cip1} and Smad6 mRNA expression were substantially increased. Knockdown of BMPRII, which is a common subunit of a functional BMPR, abolished the Smad1/5 phosphorylation and p21^{Waf1/Cip1} expression induced by LL-37, indicating that the activation of BMP signaling by LL-37 is receptor-mediated and p21^{Waf1/Cip1} expression is under the control of BMP signaling pathway in gastric cancer cells. The positive correlation between the mRNA expression of LL-37/hCAP-18 and p21^{Waf1/Cip1} in human gastric cancer tissues further supports that the expression of p21^{Waf1/Cip1} is governed, at least in part, by LL-37. Above all, the attenuation of BMP signaling by siRNA-mediated knockdown of BMPRII partially abrogated the anti-mitogenic effect of LL-37 on gastric cancer cells, further substantiating the role of BMP signaling as a negative growth-regulatory pathway (Wu, W. K., et al. (2010) J. Cell. Physiol. 223, 178-186).

In the current study, the activity of TGF-β and BMP signaling were differentially regulated by LL-37, in which the phosphorylation levels of TGF-β-responsive Smad2/3 was reduced whilst BMP-responsive Smad1/5 was increased. The phosphorylation of Smad1/5 was preceded by the induction of BMP4, suggesting that this ligand might be responsible for the activation of BMP signaling. Consistent with this finding, BMP4 has been reported to inhibit the transformed phenotype of lung adenocarcinoma cells. When treated with BMP4, these cells grow more slowly, become less invasive, and are more susceptible to apoptosis. Moreover, BMP4 and other BMPR ligands suppress the tumorigenic potential of human glioblastoma stem cells. Garrett et al. and our group have reported that BMP signaling is under the regulation of ubiquitin-proteasome system in osteoblasts as well as gastric and colon cancer cells (Wu, W. K., et al. (2008) Biochem. Biophys. Res. Commun, 371, 209-214; Garrett, I. R., et al. J. Clin. Invest, 111, 1771-1782; Wu, W. K., et al. (2008) Br. J. Pharmacol, 154, 632-638). In line with these findings, we found that LL-37 inhibits the chymotrypsin-like and caspase-like activity of proteasome. The proteasome inhibitor also mirrors the effect of LL-37 by up-regulating BMP4 expression and Smad1/5 phosphorylation. The mimicry between proteasome inhibitor and LL-37 suggests that LL-37 activates the anti-mitogenic BMP signaling via inhibition of proteasome activity (Wu, W. K., et al. (2010) J. Cell. Physiol. 223, 178-186). In relation to TGF-β signaling, the induction of Smad6, which acts as an auto-regulator of TGF-β/BMP signaling by inhibiting both pathways, may inhibit Smad2/3 phosphorylation upon activation of BMP signaling. The differential regulation of TGF-β and BMP signaling may partially explain why the expression of Smad7 was induced to a lesser extent than that of Smad6. In this respect, the transcriptional activity of Smad6 gene is predominantly regulated by BMP signaling while that of Smad7 is regulated by both. The fact that BMP signaling can induce higher p21^{Waf1/Cip1} expression than TGF-β signaling in epithelial cells may also account for why the expression of this CDK inhibitor was specifically induced by LL-37.

Cell cycle progression requires a coordinated expression of different cell cycle regulators such as cyclins, CDKs, and CDK inhibitors. Our results show that the expression of p21^{Waf1/Cip1}, which is known to inhibit the activity of cyclin E/CDK-2 complex, was induced by LL-37 with a concomitant inhibition of cyclin E₂ expression. It is known that the expression of cyclin E and the consequent increase in CDK-2 activity are essential for the progression of the cell cycle through the late G₁ phase. These findings implicated that the anti-mitogenic signaling elicited by LL-37 may pinpoint the late G₁-phase of cell cycle through dual inhibition of CDK-2 by concurrent up-regulation of p21^{Waf1/Cip1} and down-regulation of cyclin E₂. Indeed, loss of p21^{Waf1/Cip1} expression is associated with increased tumor size and lymph node metastasis of gastric cancer. Moreover, cyclin E expression also correlates with the histological grade and shortened survival in gastric cancer patients. These clinical findings together with our experimental data suggest that the loss of LL-37 expression is involved in the progression of gastric cancer.

Our present study demonstrates that the mRNA expression of LL-37 was down-regulated in gastric cancer tissues. This finding accords with the observation reported by Hase et al. that the LL-37 immunostaining was reduced in human adenocarcinomas. The mechanism by which LL-37 was down-regulated in the gastric cancer tissues, however, is unknown. In this connection, the locus 3p21, where the LL-37/hCAP-18 gene resides, is frequently deleted in gastric cancers, and many gastric cancer cell lines have homozygous deletions of 3p, suggesting that genetic mechanism may be implicated in the down-regulation of LL-37 in gastric cancer. Aside from gene deletion at the chromosomal level, dysregulation of vitamin D metabolism in gastric cancer tissues is another possible mechanism by which LL-37 is down-regulated. In relation to gastric tumorigenesis, the copy number of CYP24 gene, which encodes the enzyme responsible for the catabolism of 25-hydroxyvitamin D₃ and 1α,25-dihydroxyvitamin D₃, is increased in a large number of cases of gastric cancer. This raises the possibility that reduced de novo synthesis and increased degradation of 1α,25-dihydroxyvitamin D₃ may occur in gastric cancer, resulting in insufficient autocrine stimulation in the local tissue environment to drive the expression of LL-37.

Collectively, our experimental findings not only unveil for the first time the mechanistic pathways engaged by LL-37 to unleash its anti-mitogenic function in gastric carcinogenesis, but also shed new light on the role of innate immunity in the pathogenesis of gastric cancer.

### Example 2

### Delivery of Cathelicidin Ameliorates Colitis

Cathelicidin-knockout mice were generated by targeted disruption of Cnlp gene, which encodes the mouse cathelicidin mCRAMP. Experimental colitis was induced by dextran sulfate sodium (DSS) in Cnlp−/− and wild-type mice. The severity of colitis was assessed by clinicopathological scoring and measurement of pro-inflammatory cytokines levels and myeloperoxidase (MPO) activity. Fecal microbe population was determined by quantitative culture. Mucus secretion and mucin gene expression were measured by Periodic acid-Schiff staining and real-time PCR, respectively. Matrix metalloproteinase (MMP)-9 activity was determined by gelatin zymography. RESULTS: Cnlp−/− mice manifested more severe symptoms and mucosal disruption than the wild-type mice in response to DSS challenge. The tissue levels of interleukin-1β and tumor necrosis factor-α, MPO activity, and the number of apoptotic cells were increased in the colon of DSS-challenged Cnlp−/− mice, which harbored a larger number of aerobes and anaerobes. Moreover, mucus secretion and mucin gene expression were impaired whilst MMP-9 activity was up-regulated in Cnlp−/− mice. All these abnormalities were reversed by the intrarectal administration of mCRAMP peptide or mCRMAP-encoding plasmid. CONCLUSION: We provide in vivo evidence that cathelicidin protects against ulcerative colitis.

### Methodology

### Animals

129/VJ wild-type and cathelicidin-knockout (Cnlp^{-/-}) mice were produced as previously described (Nizet, V. et al., Nature 414:454-7 (2001)). Male (6-8-weeks old) mice were used in the following experiments. They were allowed free access to standard laboratory chow (Ralston Purina, Chicago, Ill.) and tap water. All animals were housed in an air-conditioned room with controlled temperature (22° C.±1° C.), humidity (65%-70%), and day/night cycle (12:12-h light:dark). The present study was approved by the University of Hong Kong Committee on the Use of Live Animals for Teaching and Research.

### Induction of Colitis

Acute colitis was induced in mice by giving 3% dextran sulfate sodium (DSS) (molecular weight, 36-50 kDa; ICN Pharmaceuticals, Costa Mesa, Calif.) according to a modified method by Liu et al. (Liu, E. S. et al., Carcinogenesis 24:1407-1413. Epub 2003 June 1405 (2003). The 3% DSS was given in the drinking water for 5 days (from Day 0 to Day 5). Mice receiving tap water throughout the experiment were used as a normal control group.

mCRAMP Treatment

The mice were divided into two major groups: a wild-type group and a Cnlp^{-/-} group. Each group contained a normal control group which received tap water alone and a disease group which received 3% DSS. To evaluate the protective effects of mCRAMP on ulcerative in mice, the Cnlp^{-/-} mice with colitis were further divided into three sub-groups: one without any treatment and two with mCRAMP supplements. The full length of the mature mCRAMP was purchasd from Innovagen (Lund, Sweden), and the peptide was dissolved in phosphate-buffered saline (PBS) for rectal administration. The peptide, 5 mg /kg, was administrated daily as previously described. (Tai, E. K. et al., Exp Biol Med (Maywood) 232:799-808 (2007)). The disease control mice received equal volume of PBS intra-rectally.

### Plasmid Treatment

Effective gene transfer to colonic epithelium was achieved according to the method reported by Kanbe et al. (Kanbe, T. et al., Biochem Biophys Res Commun 345:1517-1525 (2006)). A mCRAMP-expressing plasmid, pcDNA3.1/mcramp, was constructed by cloning the full-length mCRAMP cDNA into pcDNA3.1 (Invitrogen, Carlsbad, Calif.). Target gene expression was controlled under a cytomegalovirus promoter. The same plasmid without mCRAMP insert was used as negative control. All plasmids were amplified in DH5α Escherichia coli-competent cells (Invitrogen) and purified with an endo-free plasmid mega-kit (Qiagen, Valencia, Calif.). 200 μg of plasmid DNA was administrated intra-rectally at the beginning of colitis induction (i.e. Day 0). Mice were kept in an inverted position for 1 min. after administration to prevent leakage of the plasmid from the anus.

### Clinical Symptoms

For all animals, body weight, stool consistency, and presence of gross bleeding were recorded at the end of the experiment. After colon removal, the length of colon from colo-cecal junction to the anal verge was measured. Disease activity index (DAI) was calculated according to the method described by Cooper et al. (Cooper, H. S. et al., Lab Invest 69:238-249 (1993)). Briefly, body weight loss, stool consistency, and gross bleeding were scored from 0 to 4, and DAI is the combined score divided by 3.

### Morphologic Analysis

Colonic sections from all mice were fixed in 10% formalin solution (pH 7.4). Histological examination was carried out with hematoxylin-eosin staining in paraffin sections after longitudinal sections of the colon were made. The length of crypt which indicates the integrity of colon was measured as previously described (Tai, E. K. et al., Exp Biol Med (Maywood) 232:799-808 (2007)).

### Myeloperoxidase (MPO) Activity in the Colonic Tissues

Colonic MPO activity was measured as described previously (Guo, X. et al., Gastroenterology 117:884-892 (1999)). In short, colon tissue was homogenized in an ice-cold 50 mmol/ml PBS (pH 6.0) solution with 0.5% hexa-decyl-trimethyl-ammonium bromide. The homogenate was freeze-thawed three times followed by repeated sonication for 60 s each and was centrifuged for 20 min at 14000 rpm at 4° C. The activity of supernatant was determined spectrophotometrically at 450 nm. The final value was expressed as enzyme units per mg of protein.

### Fecal Microflora Count

Quantitative fecal micorflora studies were performed on freshly passed droppings. The feces collected were re-suspended in sterile PBS. After serial dilutions were done to give final concentrations of 10⁻⁴, 10⁻⁵, 10⁻⁶ and 10⁻⁷, portions of each dilution (100 μl) were spread on brain heart infusion agar (Sigma, St. Louis, Mo.) and blood agar (5% definbrinated sheep blood in Columbia agar base, Sigma) for aerobic and anaerobic microflora analysis, respectively. Plates inoculated for aerobes were incubated in air for 24 h at 37° C. Medium for anaerobe counting was incubated in an anaerobic chamber (80% nitrogen, 10% hydrogen and 10% carbon dioxide) for 48 h at 37° C. The total amount of microflora was represented as logarithms of the total number of colony forming units (CFU) found per dry weight of the stool.

### Assessment of Apoptosis

The terminal deoxynucleotidyl transferase dUTP nick-end labeling (TUNEL) method described previously was used to stain apoptotic cells (Gavrieli, Y. et al., J Cell Biol 119:493-501 (1992). The number of apoptotic cells was counted in four to six randomly selected fields at 200× magnification.

### Assessment of Mucus Thickness

Colonic samples were fixed in formalin overnight and embedded in paraffin. Five micron sections were made and stained with the periodic acid-Schiff (PAS) technique as described previously (Ma, L. et al., Gut 47:170-177 (2000)). The sections were counterstained with Harris hematoxylin and mounted in Permount (Fisher Scientific, Philadelphia, US). The mucus-containing cells were stained purple-red. The thickness of the mucus layer was measured perpendicular to the mucosal surface from the edge of the epithelium to the outermost part of the mucus layer under an image analyzer (Q500IW; Leica Image Systems) at 100× magnification. Three measurements were taken per field and approximately six consecutive fields per section were measured and results were expressed as the ratio of the thickness of the mucus layer to the thickness of the total mucosa.

### Quantitative Analysis of Mucin Gene Expression

The total RNA was isolated from mouse colonic tissues with TRIzol reagent (Invitrogen) according to the manufacturer's instructions. A total of 2.5 μg extracted RNA was used as the template for complementary DNA (cDNA) synthesis using the Thermoscript reverse transcription-polymerase chain reaction system (Invitrogen). Quantitative real-time PCR was performed for MUC1, MUC2, MUC3, MUC4, and β-actin using the primer pairs as published previously (Tai, E. K. et al., Exp Biol Med (Maywood) 232:799-808 (2007)). The cDNA was amplified using iQ SYBR Green supermix (Bio-Rad Laboratories, Hercules, Calif.) on the iCycler thermal cycler (Bio-Rad), programmed at 95° C. for 10 min, then 40 cycles of denaturation (95° C. for 15 s), annealing (59° C. for 15 s) and elongation (72° C. for 15 s). The amplification results were detected and analyzed using the iQ5 real-time PCR detection system. The gene signals were standardized against the corresponding β-actin signal and results were expressed as the ratio of each molecule to β-actin.

### Gelatin Zymography

The activity of MMP-9 was measured by zymography under non-reducing conditions (Snoek-van Beurden, P. A. et al., Biotechniques 38:73-83 (2005)). In brief, colonic tissues were homogenized (1:10) in a lysis buffer containing 50 mM Tris-HCl (pH 8.0), 150 mM NaCl, 1% NP-40, 0.5% deoxycholate and 0.1% SDS. Equivalent amounts of soluble extract were analyzed by gelatin zymography on 10% SDS-polyacrylamide gels co-polymerized with 1 mg/ml gelatin in sample buffer (10% SDS, 0.25M Tris-HCl and 0.1% Bromophenol blue, pH 6.8). After electrophoresis, gels were washed twice for 15 min in 2.5% Triton X-100, incubated for 16 hours at 37° C. in 50 mM Tris-HCl and 10 mM CaCl₂, pH 7.6, and then stained with Coomassie Blue and destained with 50% methanol. Active proteinase bands were determined by the presence of negative staining. Gel photograph were then analyzed semi-quantitatively in a multianalyzer (Bio-Rad).

### Measurement of Cytokines in Colonic Tissues

The amount of two cytokines, interleukin-1β (IL-1β) and TNF-α, in the colonic tissues was measured using commercially available enzyme-linked immunosorbent assay (ELISA) kits (Quantikine Mouse IL-1β/IL-1F2 and TNF-α/TNFSF1A Immunoassays by R&D Systems, Minneapolis, USA) according to the manufacturer's protocols. Minimum detectable concentrations were determined by the manufacturer as 3 pg/ml and 5.1 pg/ml, respectively. All samples were run in duplicate and the levels of cytokine produced in the colon were calculated as the amount per mg of protein.

### Statistical Analysis

All data were expressed as mean ±standard error (S.E.). Means were compared by the use of ANOVA and a P value of <0.05 was considered to be statistically significant.

### Results

### Cnlp^{-/-} Mice Were More Susceptible to DSS-Induced Colitis

Cnlp^{-/-} mice were used in this study to specifically address the in vivo function of mCRAMP in gastrointestinal protection. Cnlp^{-/-} mice were found to be more sensitive to DSS-induced colitis. Upon 5-day DSS treatment, Cnlp^{-/-} mice developed more severe clinical symptoms, such as gross bleeding, diarrhea, and weight loss as compared to the wild-type animals, which presented mild responses (Table 1). The DAI and crypt score representing disease severity were significantly higher in Cnlp^{-/-} mice.

mCRAMP Supplements Prevented Colitis Development

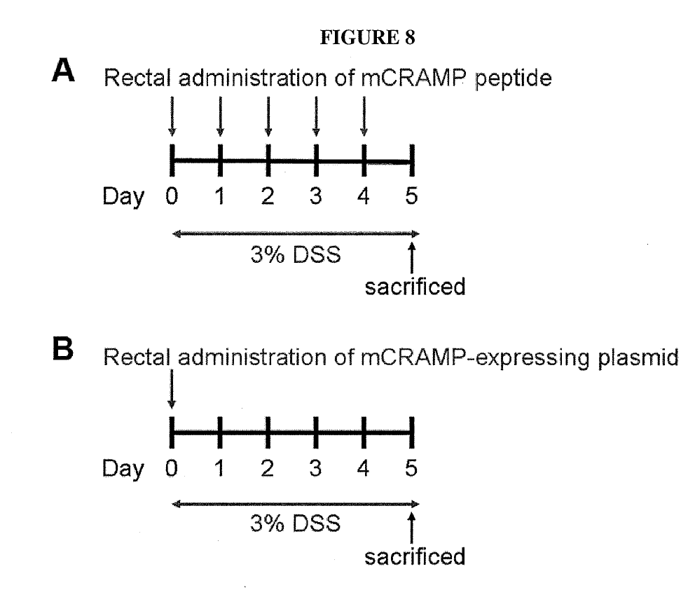

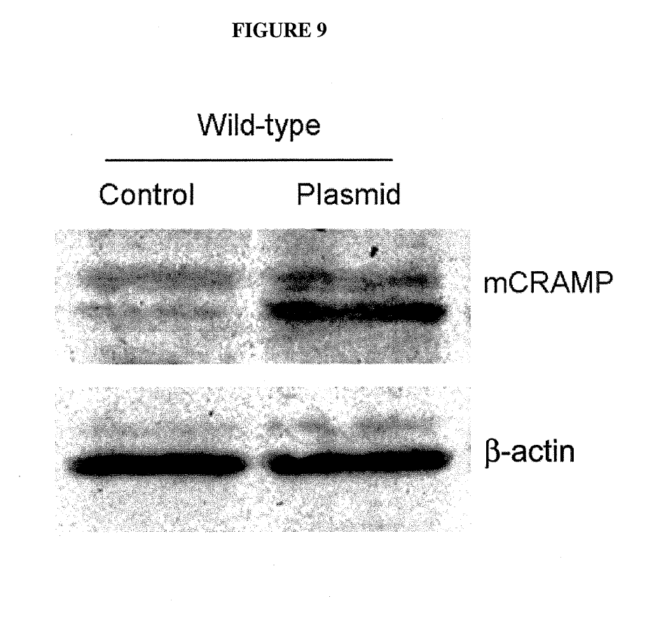

Clinical symptoms. To further examine the protective effects of mCRAMP on colitis, the synthetic peptide (5.0 mg /kg per day) or mCRAMP-expressing plasmid were administered intra-rectally to mice according to the experimental setup (FIG. 8). A single dose (200 μg) of plasmid administration effectively up-regulated the colonic expression of mCRAMP (FIG. 9). This up-regulation decreased gradually after 10 days (data not shown). Cnlp^{-/-} mice suffered from more severe colitis whereas negligible symptoms were observed in the wild-type mice (Table 1). Importantly, both mCRAMP supplements significantly ameliorate DSS-induced colitis in Cnlp^{-/-} mice. mCRAMP-treated mice showed elevated body weights, reduced disease symptoms, colonic mucosal damage, DAI and crypt scores compared with colitis Cnlp^{-/-} mice without any treatment.

TABLE 1
Effects of mCRAMP naked gene therapy
on symptoms in acute colitis

| | | 3% DSS | |
|---|---|---|---|
| | Normal control | Vector control | pcDNA3.1/mCRAMP |
| Gross bleeding | 0 | 67 | 33 |
| (% of animals) | | | |
| Loose stools | 0 | 100 | 100 |
| (% of animals) | | | |
| Diarrhea | 0 | 67 | 33 |
| (% of animals) | | | |
| Weight loss | 0 | 100 | 33 |
| (% of animals) | | | |
| Disease activity | 0.0 ± 0.00 | 2.6 ± 0.12† | 0.9 ± 0.11* |
| index (DAI) | | | |

Key:
(1) n = 3 in each group.
(2) Weight loss is defined as a ≧1% drop in body weight.
(3) †P < 0.05 compared with the normal control group.
(4) *P < 0.05 when compared with the vector control group.

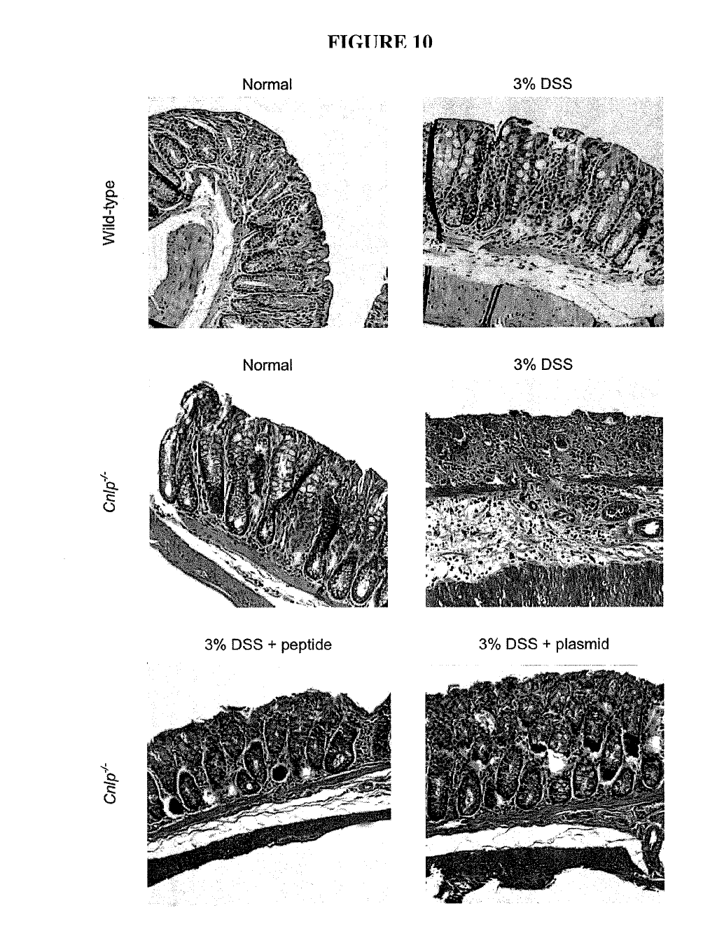

Histologic Evaluation. Results showed that there were no morphological differences in the colon between untreated wild-type and Cnlp^{-/-} mice. While DSS exerted negligible influences on wild-type mice, DSS-treated Cnlp^{-/-} mice mice showed marked signs of colonic inflammation and tissue destruction (FIG. 10). These mice displayed accumulation of red blood cells, epithelial ulceration, and a loss of crypt architecture. These were further illustrated by the distinct alteration in crypt score (Table 1). In contrast, histological analysis of the sections from mCRAMP-treated Cnlp^{-/-} mice with colitis showed obvious reduction of histological inflammation, and these animals were protected from DSS-induced mucosal injury with lesser epithelial distortion. mCRAMP supplements also increased crypt regeneration and restoration of colonic mucosa, which were comparable with the normal Cnlp^{-/-} mice.

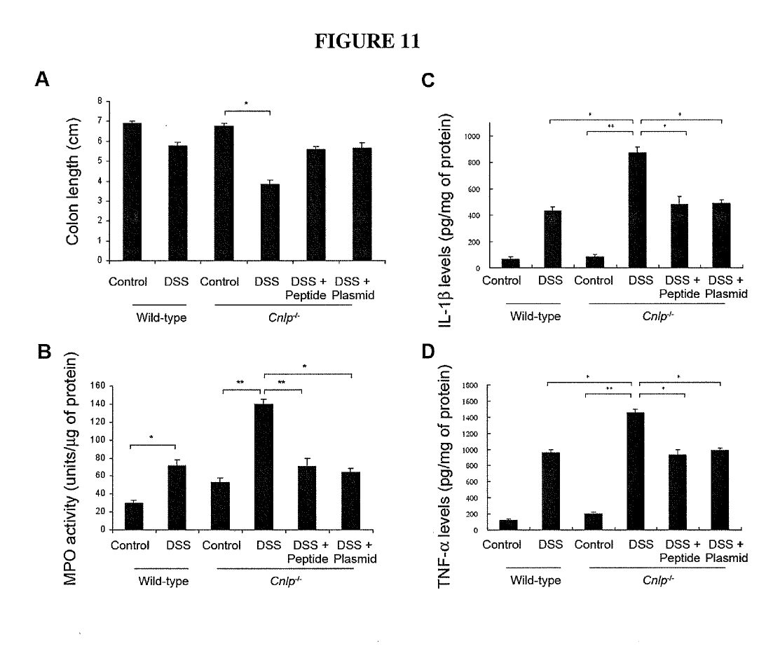

Colonic length. Reduction of colonic length, a parameter of inflammation (Okayasu, I. et al., Gastroenterology 98:694-702 (1990)), correlated with the clinical symptoms. Five-day DSS treatment showed no effect on the colonic length in wild-type mice (FIG. 11A). However, the colonic length in colitis Cnlp^{-/-} mice without mCRAMP treatment was significantly shorter than that of normal Cnlp^{-/-} mice and colitis wild-type mice. Both mCRAMP supplements effectively inhibited shortening of colon in DSS-challenged Cnlp_{-/-} mice.

MPO activity. Neutrophil infiltration into the colon was quantified by measuring the activity of MPO. The colonic MPO activity in normal Cnlp^{-/-} mice tended to be higher than the wild-type mice despite having no significance (FIG. 11B). The activity substantially increased in colitis animals in which mice displayed much higher activity than the wild-type ones. In this regard, mCRAMP supplements significantly normalized the MPO activity in colitis Cnlp^{-/-} mice to near those without DSS induction.

Colonic Cytokine Levels. The levels of two pro-inflammatory cytokines, IL-1β and TNF-α, in the colonic tissues from all tested animals were measured by ELISA. Five-day DSS treatment significantly raised the amount of both cytokines in wild-type and Cnlp^{-/-} mice (FIG. 11C & D). In particular, the levels in Cnlp^{-/-} mice increased to the greatest extent compared with the wild-type animals. This finding is similar to the disease severity in which Cnlp^{-/-} mice displayed more severe colitis symptoms. These increases were effectively negated by both mCRAMP supplements in colitis Cnlp^{-/-} mice.

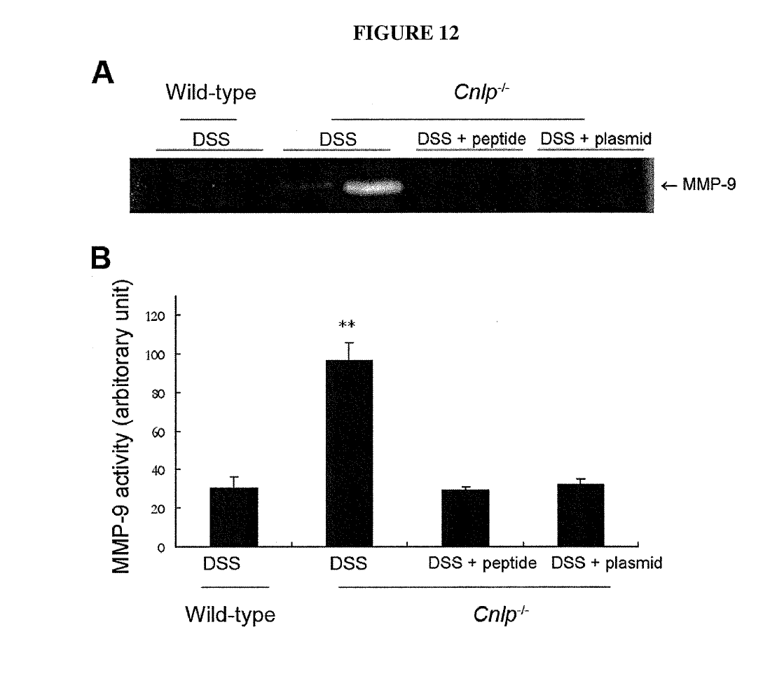

MMP-9 Activity. The colonic MMP-9 activity was analyzed in protein extracts from colitis animals using gelatin zymography. A representative zymogram and the calculated activity were shown in FIG. 12. Very low MMP-9 activity was detected in extracts prepared from colitis wild-type mice. In contrast, the colonic MMP-9 activity increased sharply in the colitis Cnlp^{-/-} mice. More than 3-fold increase was observed in colitis mice. Supplements of mCRAMP during colitis induction significantly lowered the MMP-9 activity to near the wild-type level.

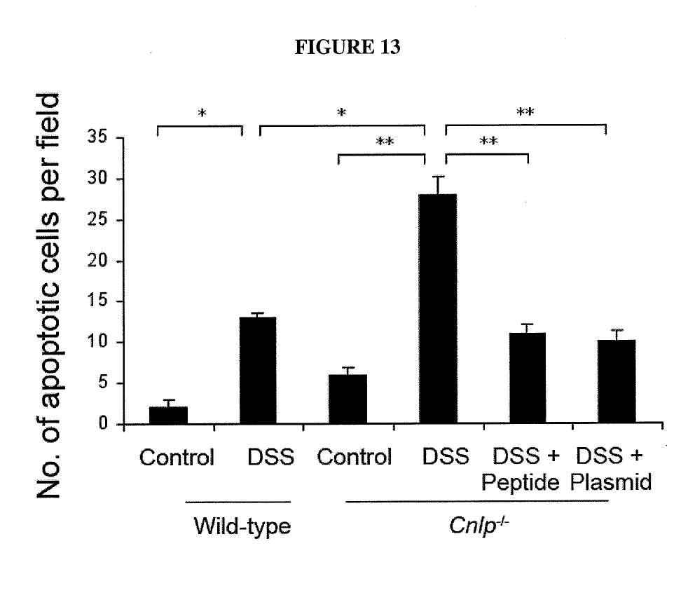

Assessment of Apoptosis. The TUNEL staining showed that the number of apoptotic cells increased significantly in both types of mice during inflammation (FIG. 13). In particular, the increase in the Cnlp^{-/-} mice was much higher than that in the wild-type animals. mCRAMP supplement substantially reduced the number of apoptotic cells back to the basal level.

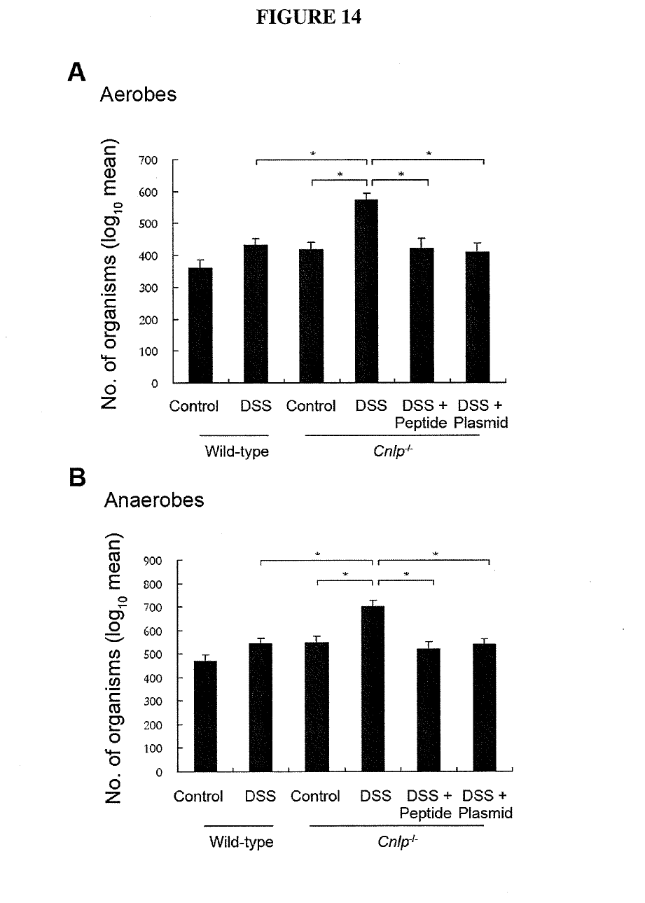

Fecal Microflora Populations. Fecal microflora examination revealed significant increases in the populations of both aerobes (FIG. 14A) and anaerobes (FIG. 14B) in Cnlp^{-/-} mice after colitis induction. mCRAMP supplements reversed these increases, and the amounts of both aerobes and anaerobes were comparable to those of the normal Cnlp^{-/-} mice mice. In the wild-type animals, although DSS treatment tended to raise the populations of fecal aerobes and anaerobes, no significant changes were detected.

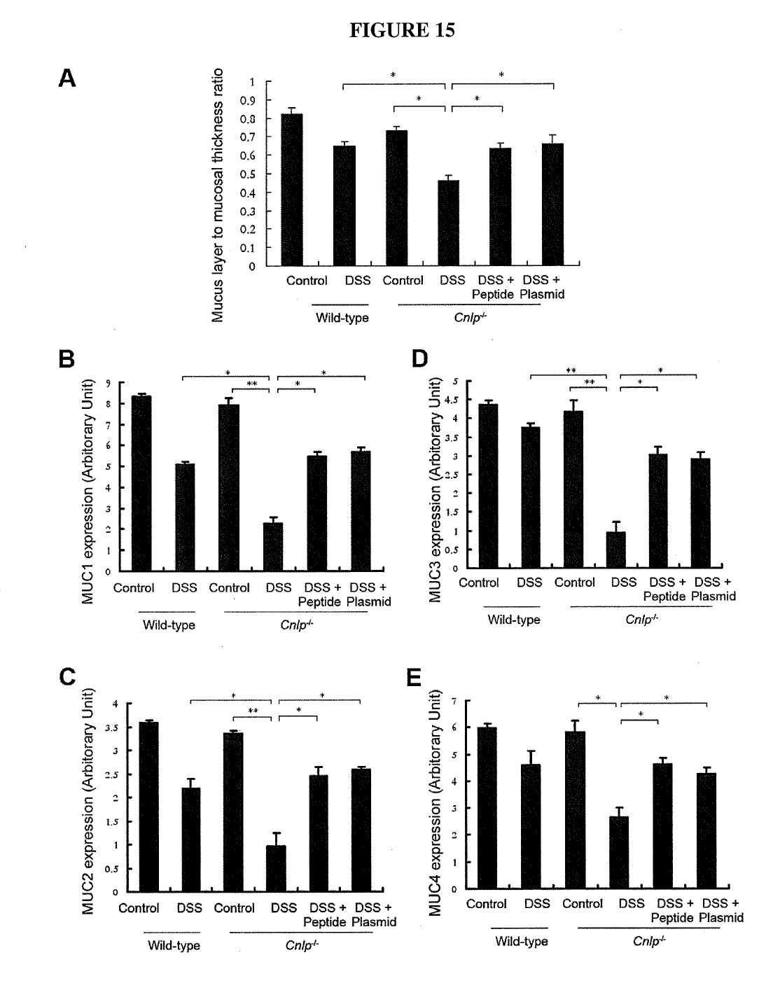

Mucus Thickness. As shown by the PAS staining, the thickness of the colonic mucous secreting layer in both wild-type and Gni' mice declined significantly during colitis (FIG. 15A). In particular, the thickness in colitis Cnlp^{-/-} mice declined to the greatest extent, −39% as compared with the normal Cnlp^{-/-} mice. Intra-rectal supplements of mCRAMP during DSS feeding markedly increased the thickness of the mucous secreting layer.

Mucin Gene Expression. The colonic gene expressions of MUC1, MUC2, MUC3, and MUC4, the building blocks of mucus, were monitored and compared among groups using real-time PCR. The results were consistent with our previous report and were summarized in FIG. 15B-E. All detected mucin genes decreased markedly during inflammation in wild-type and Cnlp^{-/-} mice. Especially, the gene expression in Cnlp^{-/-} mice was down-regulated to the greatest extent. mCRAMP supplements significantly reversed the down-regulated expression during inflammation.

### Discussion

Endogenous antimicrobial peptides, such as 13-defensin and cathelicidin, serve essential functions in the innate immune system in mammals. Cathelicidin in particular received much attention in past decades because of its pronounced influences on inflammation and wound healing (Tai, E. K. et al., Ulcer research 33:54-61 (2006). We have previously demonstrated that intrarectal administration of synthetic mouse cathelicidin could effectively mitigate experimental colitis in a murine model (Tai, E. K. et al., Exp Biol Med (Maywood) 232:799-808 (2007)). To fully address the functions of cathelicidin in gastrointestinal protection, mice with targeted deletion of Cnlp, the gene encoding the mCRAMP, and wild-type mice were evaluated for the symptomatic and histopathologic manifestations of colitis induced by DSS. The protective action of exogenous mCRAMP and mCRAMP gene transfer via an intrarectal route were also investigated in the current study.

Under normal conditions, Cnlp^{-/-} mice tended to have shorter colon length, higher MPO activity, higher populations of fecal microflora, and thinner colonic mucus than the wild-type mice although no significant differences were detected. These may partially explain the increased susceptibility towards DSS-induced colitis in the Cnlp^{-/-} mice. Histological findings indicated that deletion of Cnlp resulted in no distinct change in epithelial architecture in colon (FIG. 10). The efficiency of intrarectal mCRAMP peptide therapy has been demonstrated in our previous study (Tai, E. K. et al., Exp Biol Med (Maywood) 232:799-808 (2007)). Nevertheless, the high production cost of peptide confined the clinical application of cathelicidin in human IBDs. To overcome this problem, we have employed a novel gene delivery strategy reported by

Kanbe et al. to up-regulate mCRAMP expression in colonic epithelium (Kanbe, T. et al., Biochem Biophys Res Commun 345:1517-1525 (2006)). A mCRAMP-expressing plasmid was constructed and intrarectal administration of the plasmid could successfully boost the protein expression of mCRAMP in colon up to 10 days (FIG. 9). Results here strengthen the idea that intrarectal administration of naked DNA could be an effective therapy for human ulcerative colitis.

The present study emphasizes the importance of cathelicidin in gastrointestinal protection. Cnlp^{-/-} mice manifested significantly higher susceptibility towards DSS-induced colitis. The animals were characterized by the presence of inflammation in the colon indicated by significant weight loss, diarrhea, gross bleeding, crypt destruction, mucosal damage, and epithelial erosions. In contrast, wild-type mice only displayed mild responses after the 5-day DSS induction. It is interesting to note that both mCRAMP supplements could effectively protect Cnlp^{-/-} mice from DSS-associated mucosal injury, with mild colitis symptoms and less epithelial destruction. mCRAMP treatment alleviated neutrophil infiltration in the inflammatory colon tissues, as shown by a substantial drop in colonic MPO activity (FIG. 11). This would partly explain the protective role of mCRAMP in inflammation. In addition, apoptosis is one of the ulcerogenic process in the gastrointestinal mucosa (Liu, E. S. et al., Digestion 62:232-239 (2000)) and upsetting this process in the colonic mucosa may relate to the pathogenesis of IBDs. In this connection, mCRAMP may prevent DSS-induced colitis by inhibiting apoptosis in the colonic tissues (FIG. 13).

In past decades, the deleterious effects of enteric bacteria on IBDs were noticed. The quantity of intestinal microflora in IBD patients was higher than that in healthy subjects and increased progressively with the severity of the disease (Swidsinski, A. et al., Gastroenterology 122:44-54 (2002)). As anticipated, substantial increases in the population of both fecal aerobes and anaerobes were observed in the colitis Cnlp^{-/-} mice while mCRAMP supplements could reverse the changes although no significant change was found in the wild-type mice. This result is consistent with our previous findings that intrarectal administration of mCRAMP peptide ceased the development of colitis via abrogating the microbial increases during inflammation.

Coinciding with the previous report (Tai, E. K. et al., Exp Biol Med (Maywood) 232:799-808 (2007)), similar observations were found in the thickness of colonic mucous secreting layer. Cnlp^{-/-} mice showed significant decline in the thickness of mucus layer after colitis induction, and importantly, mCRAMP supplements preserved the mucous secreting layer and effectively reversed the reduction of mucus thickness (FIG. 14). Results from real-time PCR suggest that cathelicidin stimulates mucus synthesis in the colonic mucosa by up-regulating the mRNA expression of mucins, including MUC1, MUC2, MUC3, and MUC4. Increasing evidence suggests that abnormal mucus secretion is a pathogenic hallmark of IBD. Apparent decline in the colonic mucus thickness was associated with the initiation or perpetuation of ulcerative colitis (Corfield, A. P. et al., Infect Immun 60:3971-3978 (1992)). During the active stage of ulcerative colitis, the mucus layer is strongly disrupted and discontinuous with a 60-70% in thickness (Pullan, R. D. et al., Gut 35:353-359 (1994)). A recent MUC2-knockout mouse model has further confirmed that disruption in mucus synthesis could exacerbate experimental colitis induced by DSS (Van der Sluis, M. et al., Gastroenterology 131:117-129 (2006)). These findings substantiate the idea that cathelicidin may serve as a therapeutic tool for treating ulcerative colitis by raising mucin gene expression and enhancing mucus synthesis in the colonic mucosa, which was found to be disturbed in ulcerative colitis patients.

To further elucidate the mechanistic action of cathelicidin, we focus on the activity of MMP-9, a proteinase predominantly expressed in the gut mucosa during active colitis in the Cnlp^{-/-} and wild-type mice. MMP-9 is produced by several cell types, including macrophages, epithelial cells, and fibroblasts (Gan, X. et al., J Interferon Cytokine Res 21:93-98 (2001); McCarthy, G. M. et al., Ann Rheum Dis 57:56-60 (1998)). Intestinal epithelial cells have been shown to secrete MMP-9 in response to inflammation (Gan, X. et al., J Interferon Cytokine Res 21:93-98 (2001)). In addition, resistance to experimental colitis in MMP-9-deficient mice has pointed out the involvement of MMP-9 in intestinal inflammation (Castaneda, F. E. et al., Gastroenterology 129:1991-2008 (2005)). Results presented here illustrated for the first time that colitis Cnlp^{-/-} mice had a strong increase in colonic MMP-9 activity, and interestingly mCRAMP supplements could entirely normalize the increase (FIG. 12). It seems that cathelicidin could strengthen mucosal defense against inflammation through modulating the activity of MMP-9, which governs tissue remodeling and ulceration associated with IBDs. Alteration caused by MMP-9 activity is associated with the reduction of colonic MPO activity, which was indicative of neutrophil infiltration. Castaneda et al. have illustrated the importance of MMP-9 in neutrophil recruitment (Castaneda, F. E. et al., Gastroenterology 129:1991-2008 (2005)). MMP-9-deficient mice had minimal inflammatory infiltrates in the colonic mucosa and lamina propria. In fact, some basement membrane components are substrates for MMP-9 and it has been proposed that MMP-9 could facilitate the emigration of neutrophils.

It is established that pro-inflammatory cytokines play a pivotal role in the inflammation of the intestinal mucosa (Bouma, G. et al. Nat Rev Immunol 3:521-533 (2003); Pullman, W. E. et al., Gastroenterology 102:529-537 (1992)). Elevated levels of various cytokines have been detected in mucosal tissue samples from patients with IBDs (Murata, Y. et al., J Gastroenterol 30 Suppl 8:56-60 (1995)). They contribute to the increased migration of neutrophils and monocytes into the lesion of IBDs and to the activation of inflammatory cells. Particularly, IL-1β, IL-6, and TNF-α have been proposed to take part in the pathogenesis of IBDs (Fiocchi C. Gastroenterology 115:182-205 (1998); Stevens, C. et al., Dig Dis Sci 37:818-826 (1992)). These cytokines result in amplification of the inflammatory cascade and secretion of more inflammatory mediators, destructive enzymes, and free radicals that cause tissue injury (Sartor, R. B. Gastroenterology 106:533-539 (1994)). Analysis of colonic tissues of wild-type and Cnlp^{-/-} mice showed remarkable increases in the level of IL-1β and TNF-α during DSS-induced colitis. Most notably, the increases in Cnlp^{-/-} mice were about 40-50% higher than the wild-type mice. Consistent with the above observation, mCRAMP supplements could effectively eliminate the alterations in colonic cytokine levels. Real-time PCR data showed no observable changes in IL-6 level in the mice (data not shown). In conjunction, these results support our hypothesis that cathelicidin could relieve intestinal inflammation through modulating the secretion of pro-inflammatory cytokines.

The modulatory effect of cathelicidin on cytokines may in addition explain its inhibitory action on MMP-9 activity. Mounting evidence revealed that pro-inflammatory cytokines increases MMP production (Mott, J. D. et al., Curr Opin Cell Biol 16:558-564 (2004)). TNF-α substantially stimulates MMP-9 expression in human monocytes through the activation of mitogen-activated protein kinases. Constitutive MMP-9 secretion is also abolished by incubation of neutralizing antibody against TNF-α (Heidinger, M. et al., Biol Chem 387:69-78 (2006)). Xie et al. furthermore showed that IL-1β consistently stimulated MMP-2 and MMP-9 activity in adult rat cardiac fibroblasts (Xie, Z. et al., J Biol Chem 278:48546-48552 (2003)). The cytokine data here seems to imply the indirect action of cathelicidin on MMP-9 activity. Cathelicidin probably interferes with the secretion of IL-1β and TNF-α, and eventually results in the reduction of MMP-9 activity.

The findings presented so far provide direct evidence on the protective role of cathelicidin against colonic inflammation. Cnlp^{-/-} mice displayed increased susceptibility towards DSS-induced colitis and cathelicidin supplement or gene therapy could efficiently alleviate intestinal inflammation. mCRAMP-treated colitis mice showed more mild inflammatory responses with reduced clinical symptoms and lesser mucosal damage. Apart from the antimicrobial action and stimulatory effects on colonic mucus synthesis, we further demonstrated its inhibitory action on pro-inflammatory cytokine secretion as well as MMP-9 activity in the colon. Cathelicidin may prevent colitis development by blocking the deleterious inflammation cascade of cytokines and by reducing extracellular matrix degradation by MMP-9. We have previously reported that repeated cycle of injury and repair in the intestinal mucosa could increase the risk for colon cancer (Liu, E. S. et al., Carcinogenesis 24:1407-1413. Epub 2003 June 1405 (2003)). Prophylactic treatment of ulcerative colitis could possibly overcome the risk of cancer development in IBD patients. The multiple actions of cathelicidin render it as a prophylactic therapy for IBDs and colitis-associated colon cancer.

### Example 3

### Delivery of Lactoccocus Expressing Cathelicidin Ameliorates Inflammation and Colitis

The present invention provides an agent for treating multi-factorial and bacteria-related diseases in the gastrointestinal tract. Cathelicidin is an antibacterial peptide that exists endogenously at the epithelium of the gastrointestinal tract but is deficient in cancer tissues and up-regulated in inflammatory tissues. Here, we describe use of genetic engineering technology to transduce the probiotic Lactoccocus lactis with the DNA fragment encoding cathelicidin to prolong the expression of this functional peptide in the gastrointestinal tract. This multi-targeted preparation is useful for, e.g., the prevention and treatment for inflammatory and cancerous disorders in the gastrointestinal tract.

We constructed and developed a cathelicidin-secreting probiotic, namely Lactococcus lactis using the following biotechnology technique.

Nisin-Controlled Secretion of Cathelicidin in Lactococcus lactis

### Recombinant Plasmid Construction

The DNA fragment encoding cathelicidin (LL-37 or mouse cathelicidin as discussed more below) was chemically synthesized based on bias codons of Lactococcus lactis with two restriction enzyme sites at the 5′- and 3′- ends, respectively. We used the DNA fragments encoding human cathelicidin (LL-37) and mouse cathelicidin (mCRAMP) to be incorporated into the codons of Lactococcus lactis. For the final recombinant plasmid pNZ8149-usp-Cath, either the LL-37 or mCRAMP was used for the determination of LL-37 synthesis in the bacteria or for the study of ulcerative colitis in mouse, respectively. To produce secretory cathelicidin in food-grade lactic acid bacteria Lactococcus lactis, the DNA fragment encoding the signal peptide of usp45 gene and the nine-residue propeptides LEISSTCDA (LEISS) was incorporated immediately upstream to cathelicidin. The DNA fragment was cloned into the nisin-controlled gene expression (the NICE system) vector pNZ8149 to generate the recombinant plasmid pNZ8149-usp-Cath. The recombinant plasmid was confirmed by DNA sequencing.

Nisin Controlled Secretory Expression of Cathelicidin in Lactococcus lactis

The pNZ8149 plasmid and Lactococcus lactis was purchased from NIZO Food Research B.V. (The Netherlands). pNZ8149 (empty vector) is a vector with the cat-gene replaced by the foodgrade lacF gene as selection marker. It was used in combination with L. lactis NZ3900. We used the host strain of Lactococcus lactis NZ3900, which is based on the Model strain MG 1363 with nisR and nisK genes integrated in the chromosome. It is the host strain for nisin inducible vectors. There is a lactose operon on the chromosome with a deletion in the lacF gene. It can be used for foodgrade overproduction in combination with vector pNZ8149. We transformed PNZ8149-usp-Cath and pNZ8149 plasmids into Lactococcus lactis NZ3900, respectively. pNZ8149 was used as empty vector control.

The pNZ8149-usp-Cath plasmid was transformed into food-grade Lactococcus lactis NZ3900 by electroporation. The positive clones of NZ3900 containing pNZ8149-usp-Cath plasmid were screened by PCR. We obtained two positive clones as confirmed by PCR and enzyme digestion. Five milliliters of NZ3900 bacteria containing pNZ8149-usp-Cath recombinant plasmid were cultured at 30° C. overnight. The transduced Lactococcus lactis was then diluted 1/25 in 2×50 ml fresh M17 medium, incubated at 30° C. until OD₆₀₀=0.4-0.5, and 10 ml of the culture was induced with 10 ng/ml nisin (Sigma) for another 3 hours. The other 10 ml was used as a negative control. Cell were then centrifuged at 4500×g for 15 min and the supernatant was transferred to a new 50 ml tube. Fifteen milliliters of the supernatant was added to the amicon ultra-15 filter unit (Millipore, ULTRACE-3K). The capped filter device was placed into the swinging bucket centrifuge rotor and spun at 4000×g for 45 minutes. A pipetter was inserted into the bottom of the filter unit and the sample was withdrawn into a 1.5 ml Eppendorf tube. The volume left was about 500 μl.

### Detection of Secreted Cathelicidin by ELISA

The amount of cathelicidin peptide in the supernatants and concentrated samples of the induced culture and negative control were detected by ELISA (Human LL-37 ELISA TEST KIT, Hbt HK321, Hycult Biotechnology (HBT), Netherlands)) according to the users' manual. The cathelicidin concentrations of 30× concentrated samples were as following: Clone 2-1:570 pg/ml; Clone 2-2:540 pg/ml. The cathelicidin concentrations of supernatant were: Clone2-1:58 pg/ml, Clone 2-2:48 pg/ml.

L. lactis Expressing LL-37 Decrease Colitis in Mice

Male Balb/c mice 6-8 weeks old were used in the following experiments. They were allowed free access to standard laboratory chow and tap water. All animals were housed in an air-conditioned room with controlled temperature (22° C.±1° C.), humidity (65%-70%), and day/night cycle (12:12-hr light: dark). Mice were induced with acute colitis by giving 3% dextran sulfate sodium (DSS, molecular weight, 35-50 kDa) prepared in drinking water for 7 days (from Day 0 to Day 7). Normal control mice received tap water throughout the experiment. Lactococcus lactis NZ3900 or NZ3900 transformed with mouse cathelicidin were incubated in M17 broth with 0.5% glucose and 0.5% lactose respectively at 30° C. without aeration overnight, then diluted in fresh broth in 1:25 ratio, and incubated until OD600 reached 0.4-0.5. In a different group of transformed NZ3900, 0.25 ng/ml nisin was added and further incubated for 3 h. Bacteria were then harvested by centrifugation (5000 rpm, 15 min) and washed twice with sterilized PBS (pH 7.4) and resuspended in sterilized water to give 2×10¹⁰ cfu/ml. Mice were given intragastrically (i.g.) with 0.5 ml (1×10¹⁰ cfu) bacteria once daily at the beginning of the DSS feeding. Animals were sacrificed at the end of 7 days DSS drinking and bacteria feeding.

Colonic myeloperoxidase (MPO) was measured as described previously (Guo X, et al. Gastroenterology 117, 884-892 (1999)). In brief, colon tissues were homogenized in an ice-cold 50 M PBS (pH 6.0) solution with 0.5% hexa-decyl-trimethyl-ammonium bromide. The homogenate was freeze-thawed three times, followed by repeated sonication for 60 seconds each and centrifugation for 20 minutes at 14,000 revolutions per minutes at 4° C. The activity of the supernatant was determined spectrophotometrically at 450 nm. The final value was expressed as enzyme units per mg of protein.

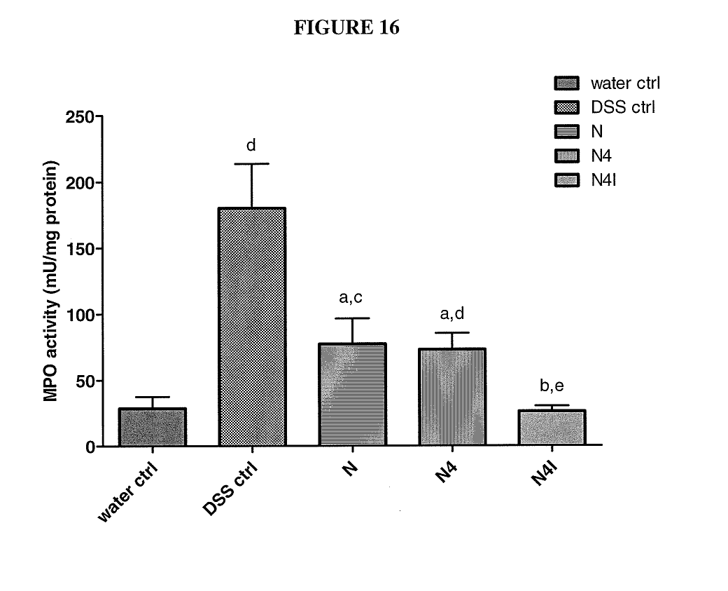

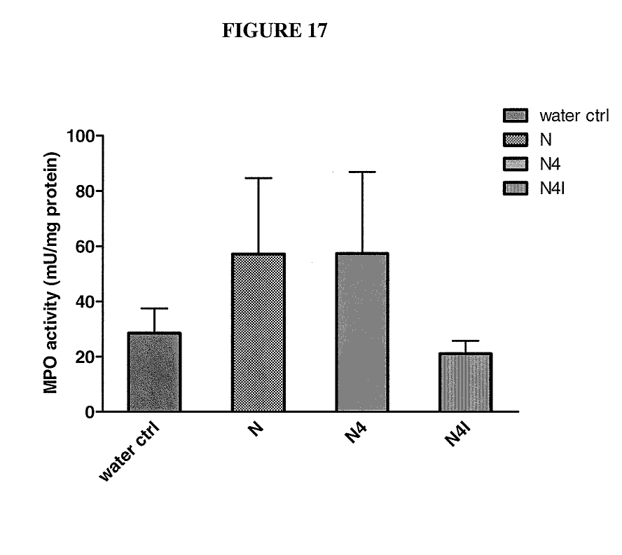

Oral administration of Lactococcus lactis with cathelicidin transfection together with nisin induction protected against dextran sodium sulfate-induced ulcerative colitis in mice. Results of the MPO assay are shown for two sets of experiments in FIG. 16 and FIG. 17. The effect was found to be significant and dose-dependent (from 10⁸ to 10¹⁰ CFU). The most effective dose was noted to be 10¹⁰ CFU given once daily for 7 days. It is expected that 10 times higher than this dose would be effective for ulcerative colitis in humans.

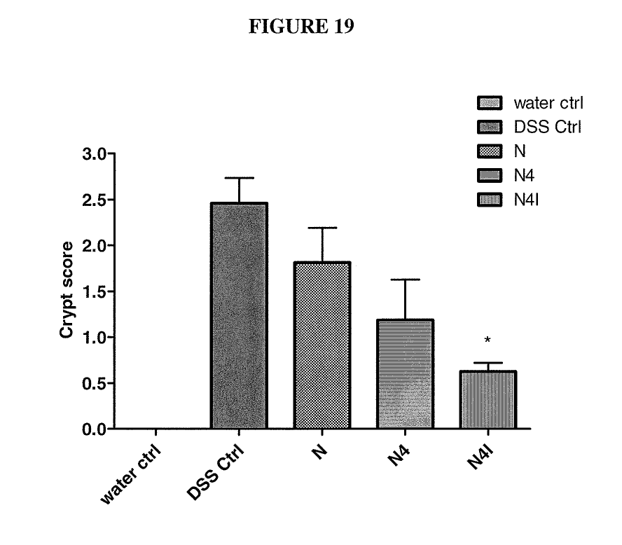

Crypt loss is a histological finding in colitis. Therefore, histological analysis of mice administered with formulations per FIG. 16. The crypts were graded on a 4 grade scale as follows: Grade 0: intact crypt; Grade 1: loss of the basal ⅓ of the crypt; Grade 2: loss of the basal ⅔ of the crypt; Grade 3: loss of entire crypt with surface epithelium remaining intact; Grade 4: loss of both the entire crypt and surface epithelium. FIG. 19 shows the results of the analysis and shows that administration of bacteria induced to express cathelicidin significantly reduced crypt loss compared to the control.

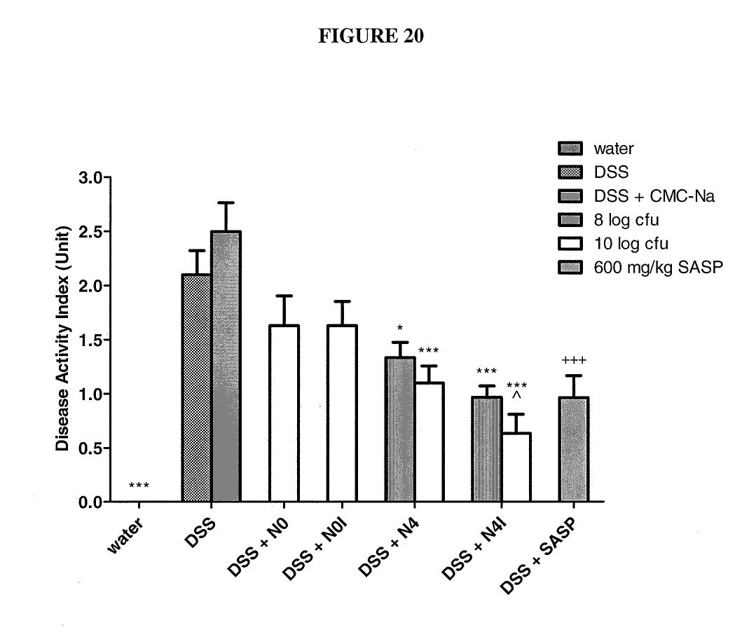

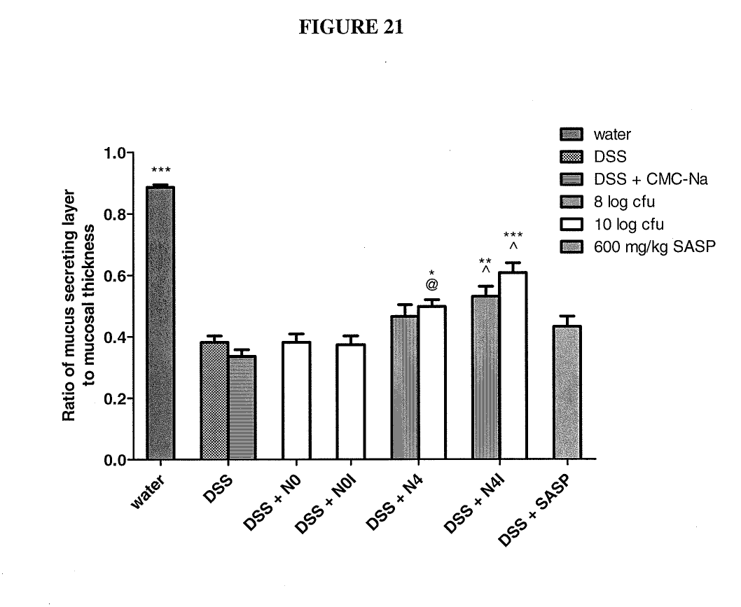

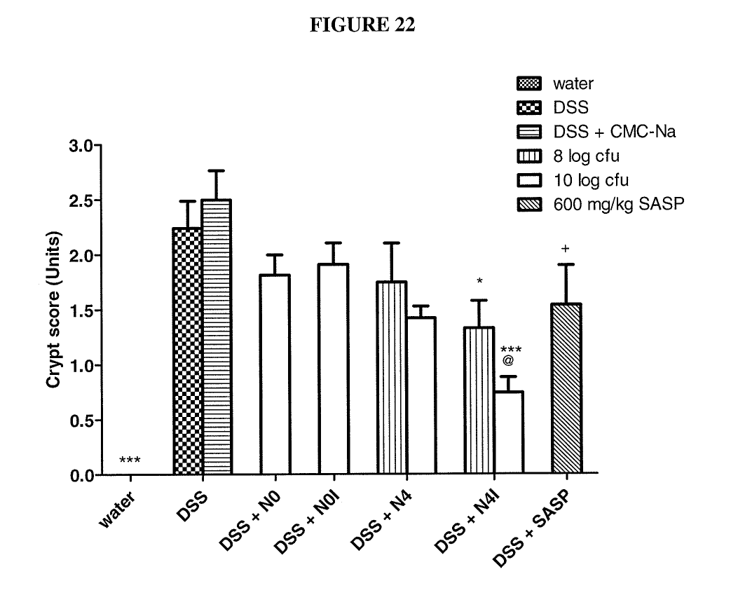

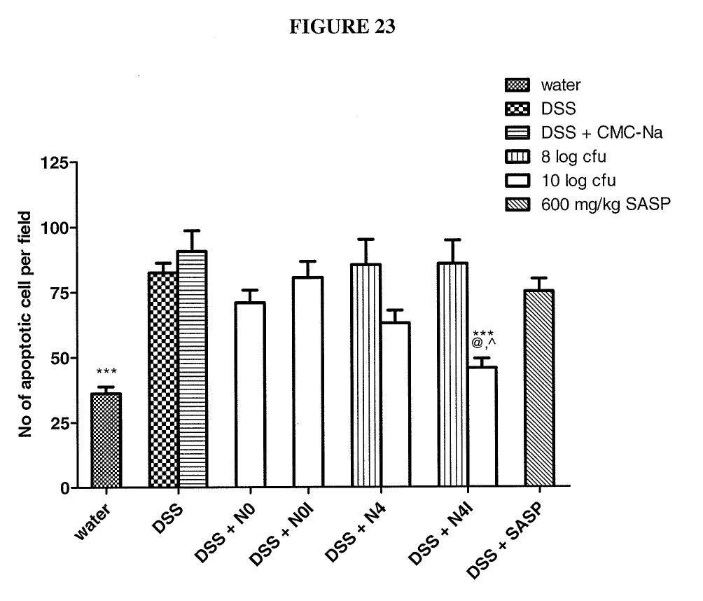

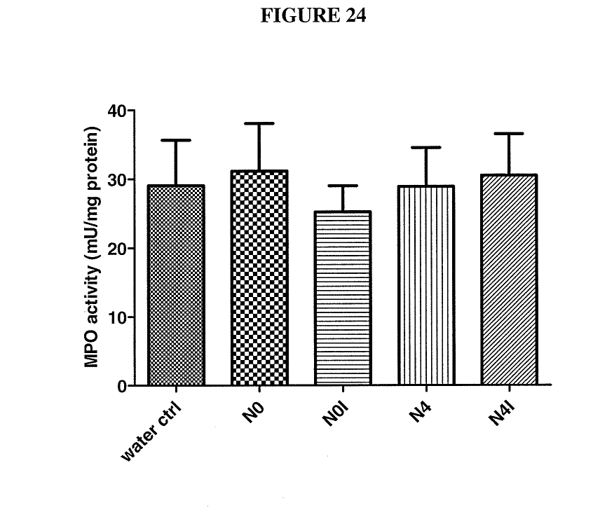

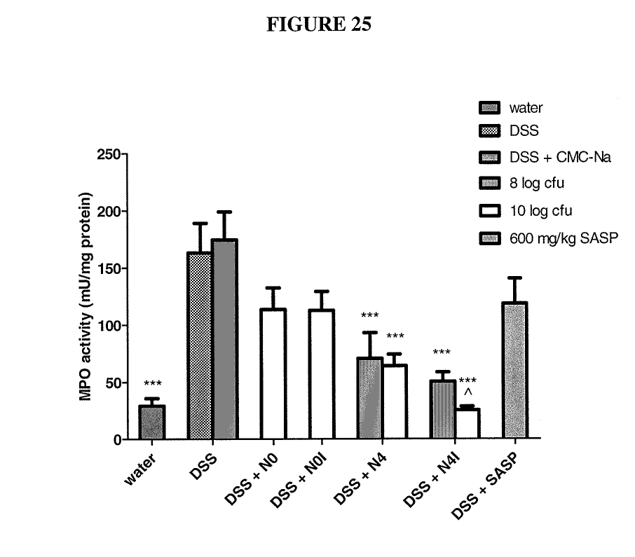

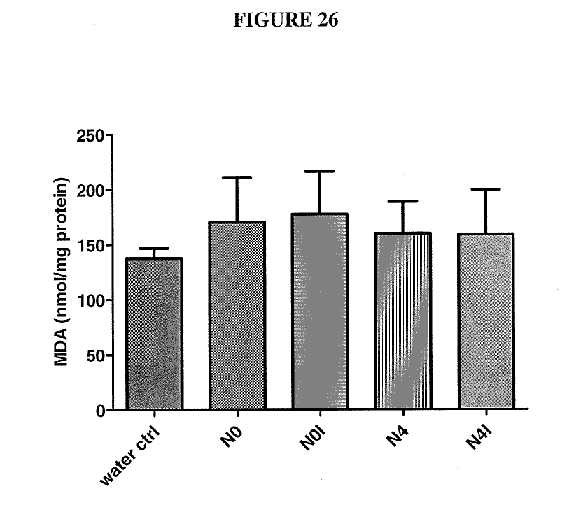

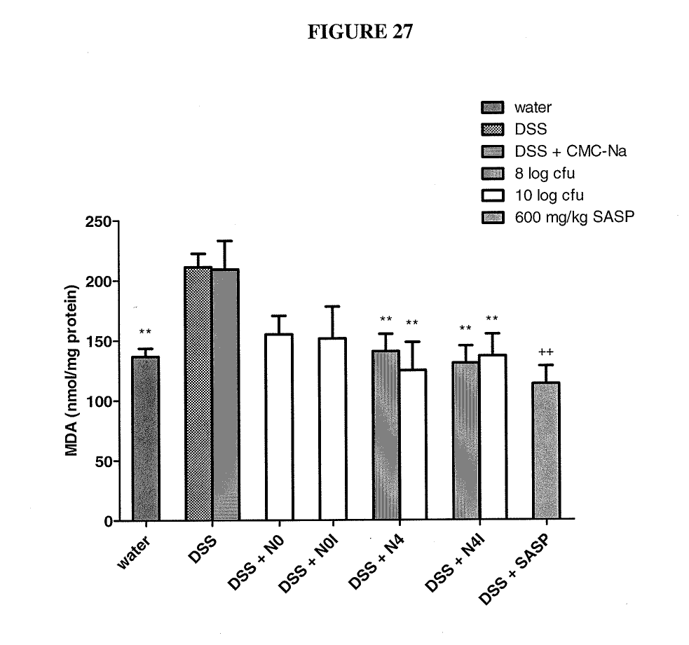

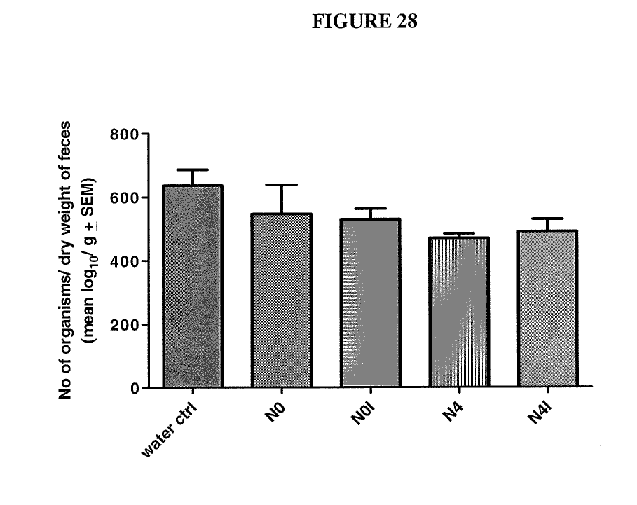

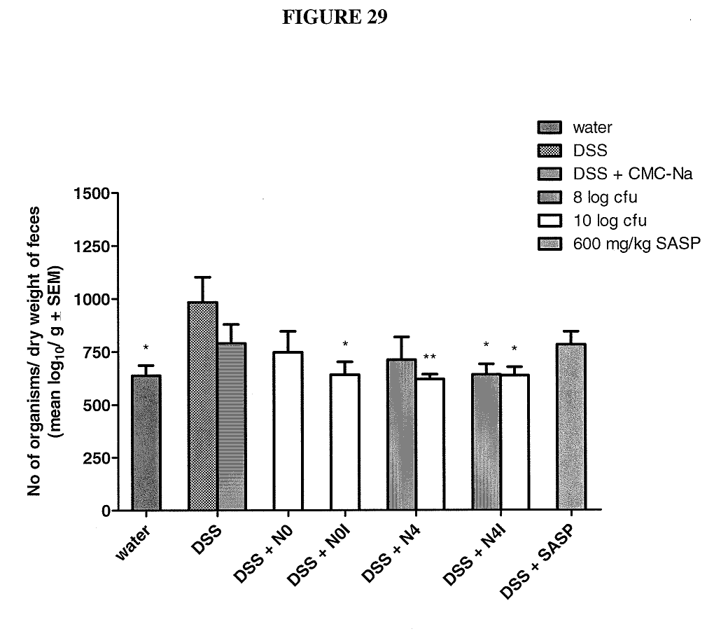

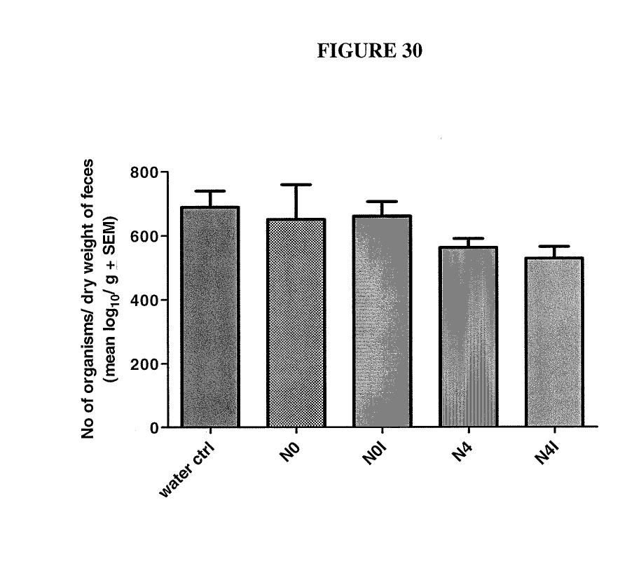

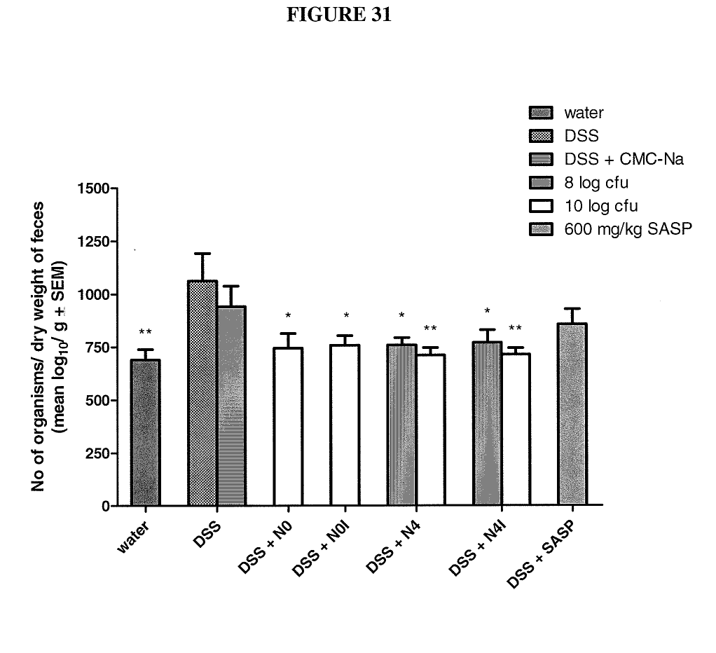

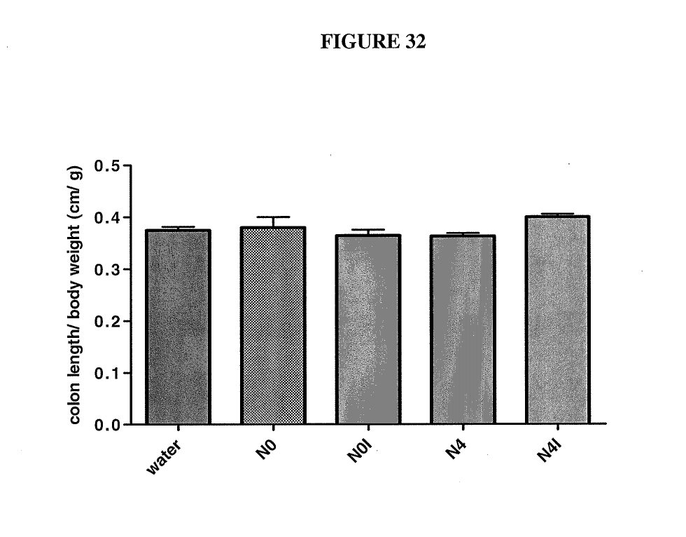

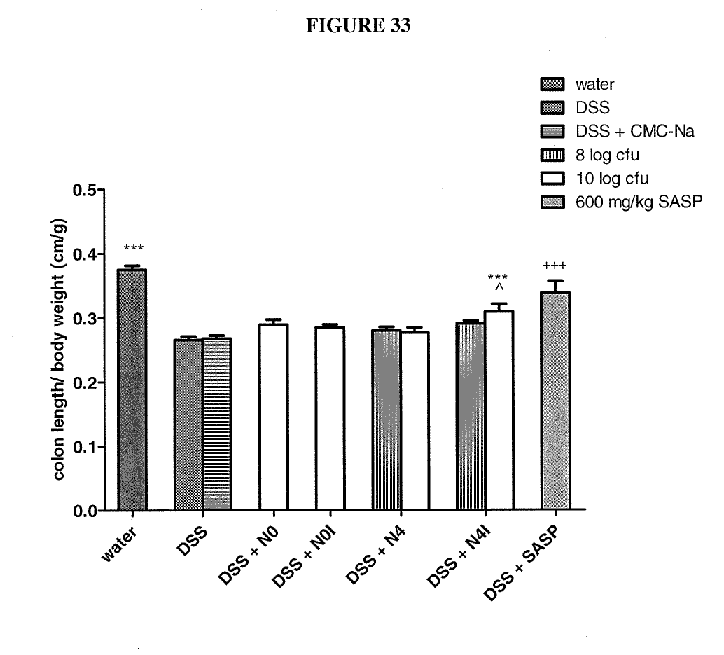

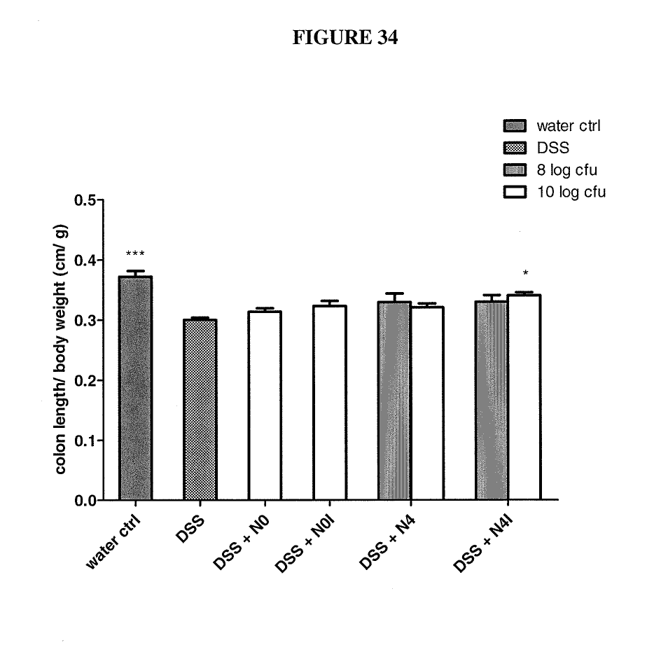

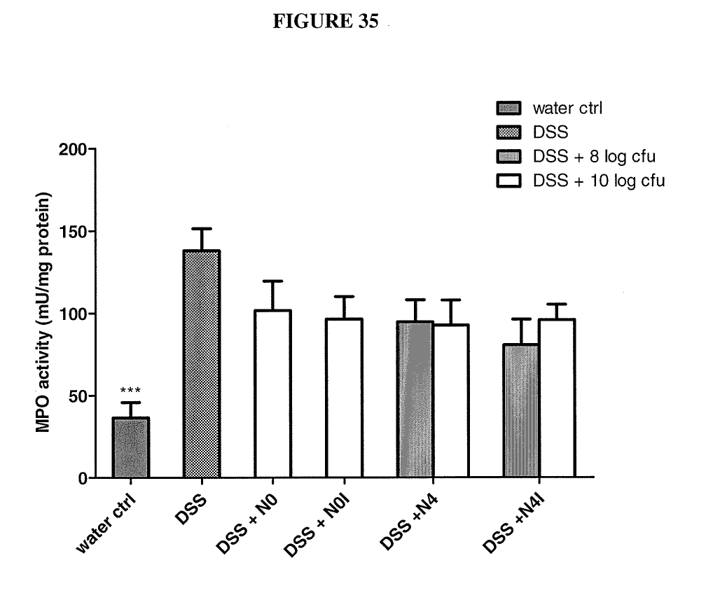

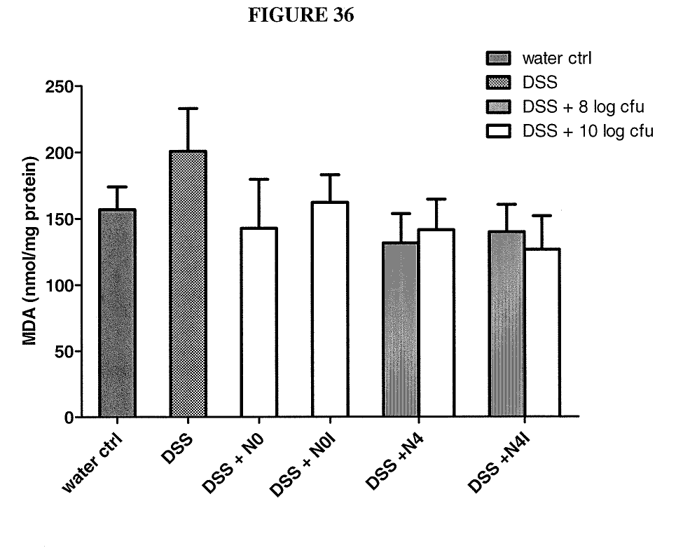

These experiments were repeated in more detail using oral administration of Lactococcus lactis with cathelicidin transfection together with nisin induction to examine protection against dextran sodium sulfate-induced ulcerative colitis in mice. The results of the experiments can be found in FIGS. 20-36 and can be summarized as follows:
1. Lactococcus lactis NZ3900 with or without transformed mCRAMP did not adversely affect the colonic mucosa and number of microorganisms in the colon when give orally once daily for 7 days in mice.
2. DSS given in drinking water for 7 days however induced human-like ulcerative colitis in mouse. DSS increased the disease activity (body weight drop, diarrhea and bloody stool), crypt damage, mucus depletion, neutrophil infiltration (reflected by increased MPO activity), lipid peroxidation (reflected by increased MDA level) and apoptotic cells in the colonic mucosa, and also microorganism number in the feces. DSS also decreased the colon length over body weight ratio, a typical and good indicator for inflammation in the colon
3. Lactococcus lactis NZ3900 transfected with mCRAMP in particular with nisin induction significantly reversed all the actions above. All these findings indicate that probiotic with transformed cathelicidin may have therapeutic potential for the prevention of ulcerative colitis.
4. Sulfasalazine a prototype drug for ulcerative colitis produced the similar preventive effects. However, unlike Lactococcus lactis encoded with cathelicidin, it did not affect the number of microorganisms in the colon, MPO activity, mucus level, and number of apoptotic cells in the colonic mucosa. These findings suggest that Lactococcus lactis has better therapeutic effects than sulfasalazine in the treatment of ulcerative colitis.
5. DSS administration continued to decrease the colon length/body weight ratio (an indicator for inflammation in the colon) after stopping DSS for 4 days. Lactococcus lactis NZ3900 transfected with mCRAMP plus nisin induction treatment for 4 days significantly reversed this effect.
6. DSS also continued to increase the MPO activity 4 days after stopping DSS administration but the effect was unaffected by all preparations tested.
7. The increase of MDA level after termination of DSS administration for 4 days was insignificant. Again this was unaffected by the preparations tested in this study.

### Example 4

### Delivery of Lactoccocus Expressing Cathelicidin Ameliorates Gastric Cancer

### Gastric Cancer Induction

MKN-45 cancer cells were trypsinized, collected and resuspended in phosphate buffered saline (PBS, pH=7.2) on ice. Then 1×10⁷ cells in 0.2 mL were injected subcutaneously into the left axillary fossa region of 4-6-week-old female BALB/c nu/nu mice. After a second passage culture in vivo, the subcutaneous tumors were removed aseptically, cut into pieces of about 1 mm³ and kept in PBS on ice. In the recipient mouse, the serous layer of the greater curvature of stomach with abundant blood vessels was carefully ruptured with a grater until bleeding was visible and a piece of tumor was implanted. Animals were kept in a sterile environment.

### Probiotic Treatment

Seven days after implantation, mice were randomized into two groups: control group received 0.25 mL distilled water and the treatment group received 0.25 mL of 1×10¹⁰ CFU/mL L. lactis NZ3900 transformed with human cathelicidin LL-37 which had been incubated with 0.25 ng/mL nisin for 3 hours (LL-37-encoding L. lactis). Mice were treated with these preparations by oral route every other day for two weeks. At the end of the experiment, mice were sacrificed and tumors were excised for further assays.

### Results:

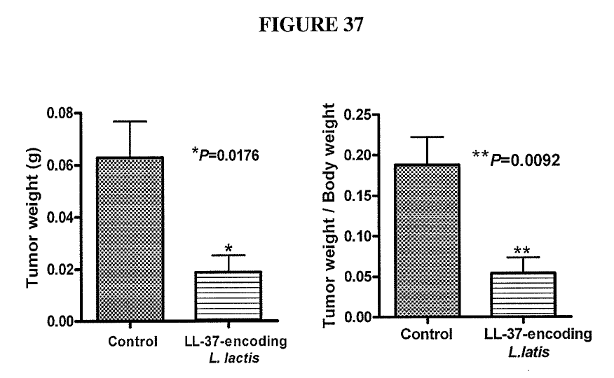

Treatment with Lactococcus lactis encoded with LL-37 and nisin induction for two weeks significantly reduced tumor growth by 70%. See, FIG. 37.

### SUMMARY

The ulcer healing action of cathelicidin on rat stomachs has been established both in vitro and in vivo, including the mechanisms of action through the TGFa and EGF receptors. The anti-inflammatory actions in mouse colons have been defined, including the mechanisms to enhance mucus secretion and reduce inflammatory cytokines, such as TNF-α and IL-1β, increase in anti-inflammatory cytokine, e.g. IL-10 both in vitro and vivo. The importance of cathelicidin in inflammatory bowel disease was confirmed in cathelicidin knock-out mice in which the disease is markedly aggravated. The anti-cancer action in human gastric cancer cells was also found and the signal transduction pathway was defined through the bone morphogenetic protein (BMP) pathway.

Cathelicidin-coding DNA was introduced into a plasmid which was used to prolong the expression of the peptide in gastric and colon tissues for at least for 7 days, resulting in ulcer healing and anti-inflammatory action in the stomach and colon. Lactococcus lactis was found to survive and identify in the gastric and colonic mucosae and lumen. Therefore, incorporation of cathelicidin into Lactococcus lactis as one single preparation has been successfully established. As cathelicidin is constitutively expressed in the body and probiotic is consumed in humans for nutritional use for disorders in the gastrointestinal tract, this cathelicidin-expessing probiotic is expected to be safe and can be given orally for chronic diseases such as inflammation (including but not limited to colitis) and cancer in the gastrointestinal tract.

Our previous study showed that cathelicidin given intrarectally alleviated the inflammatory responses induced by dextran sulfate sodium in mouse colons, through the reduction of inflammatory cytokines (TNF-α and IL-1β), MMP-9 and increase in anti-inflammatory cytokine (IL-10), and mucus production. It also reduced bacterial overgrowth in the colon. In cathelicidin knock-out mice, the inflammation was worsened but prevented by cathelicidin-encoded plasmid. In addition, oral administration with cathelicidin encoded-lactococcus lactis produced the similar protective effect against inflammation in the colon. As inflammation in the colon is positively related with colorectal cancer, we propose that cathelicidin-expressing Lactococcus lactis is useful for the treatment of inflammatory bowel diseases and colorectal cancer.

Cathelicidin as an antibacterial peptide reduced human gastric cancer cell proliferation in vitro, via activation of BMP signaling tumor-suppressing pathway. Cathelicidin-encoded Lactococcus lastis reduced gastric tumor growth in mice. All theses findings support the usage of cathalicidin-encoded Lactococcus lactis as a therapeutic agent for the treatment of gastric cancer, for example, as related to Helicobacter pylori infection.

Cathelicidin-encoding plasmid promoted gastric ulcer healing in rat stomachs by enhancing cell proliferation and angiogenesis. The peptide directly stimulated gastric epithelial cell proliferation through transforming growth factor and epidermal growth factor receptor activation in isolated rat gastric epithelial cells. Cathelicidin-expressing Lactococcus lactis is thus useful for gastric ulcer healing.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A lactic acid bacteria transformed to secrete biologically active cathelicidin.

2. The bacteria of claim 1, wherein the lactic acid bacteria is selected from the group consisting of Lactococcus sp., Lactobacillus sp., and Bifidobacterium sp.

3. (canceled)

4. The bacteria of claim 1, wherein expression of the cathelicidin is under the control of an inducible promoter.

5. The bacteria of claim 1, wherein the bacteria comprises a nucleic acid coding sequence for the cathelicidin and the coding sequence has been codon-improved for the species of the bacteria.

6. The bacteria of claim 1, wherein the cathelicidin is at least 80% identical to SEQ ID NO:1 or SEQ ID NO:2.

7. A method of reducing inflammation in the gastrointestinal tract of a mammal, the method comprising administering an amount of the lactic acid bacteria of claim 1 sufficient to reduce inflammation in the gastrointestinal tract of the mammal.

8. The method of claim 7, wherein 10⁸ to 10¹² CFU of the bacteria are administered to the mammal daily.

9. (canceled)

10. The method of claim 7, wherein the bacteria is administered for at least three days.

11-12. (canceled)

13. The method of claim 7, wherein the lactic acid bacteria is selected from the group consisting of Lactococcus sp., Lactobacillus sp., and Bifidobacterium sp.

14. The method of claim 7, wherein the mammal has colitis or bowel inflammatory disease.

15. The method of claim 7, wherein the mammal has Crohns' disease.

16. The method of claim 7, wherein the mammal has colorectal or gastric cancer.

17. The method of claim 7, wherein the mammal has a condition selected from the group consisting of: gastritis, gastric ulcers or gastroesophageal reflux disease (GERD).

18-21. (canceled)

22. The method of claim 7, wherein the cathelicidin is at least 80% identical to SEQ ID NO:1 or SEQ ID NO:2.

23. (canceled)

24. A food product comprising an amount of the lactic acid bacteria of claim 1 sufficient to reduce inflammation in the gastrointestinal tract of a mammal.

25-27. (canceled)

28. An isolated nucleic acid comprising a nucleic acid coding sequence for a biologically active cathelicidin, wherein the coding sequence has been codon-improved for expression in a lactic acid bacteria.

29. The nucleic acid of claim 28, further comprising a promoter operably linked to the coding sequence.

30. The nucleic acid of claim 29, wherein the promoter is an inducible promoter.

31. The nucleic acid of claim 28, wherein the coding sequence comprises at least one codon improved for expression in Lactococcus sp., Lactobacillus sp., or Bifidobacterium sp.
